(19) United States
(12) Patent Application Publication
(10) Pub. No.: US 2021/0254044 A1
(43) Pub. Date: Aug. 19, 2021

(54) METHOD FOR CAPTURING AND ENCODING NUCLEIC ACID FROM A PLURALITY OF SINGLE CELLS

(71) Applicant: Fluidigm Corporation, South San Francisco, CA (US)

(72) Inventors: Sten Linnarsson, Stockholm (SE); Gioele Le Manno, Stockholm (SE); Amit Zeisel, Stockholm (SE)

(73) Assignee: Fluidigm Corporation, South San Francisco, CA (US)

(21) Appl. No.: 17/065,922

(22) Filed: Oct. 8, 2020

Related U.S. Application Data
(63) Continuation of application No. 15/025,874, filed on Mar. 29, 2016, now abandoned.

(30) Foreign Application Priority Data
Sep. 30, 2013 (GB) 1317301.8

(51) Int. Cl. C12N 15/10

(52) U.S. Cl. CPC C12N 15/1006; C12Q 2563/185; C12N 15/1065; C12N 15/1096

(57) ABSTRACT

This invention relates to methods for capturing and encoding nucleic acid from a plurality of single cells. A plurality of solid supports is randomly placed into a plurality of compartments, such that the average number of solid supports per compartment, λ₁, is less than 1, wherein each solid support carries (a) a unique identification sequence and (b) a capture moiety. A plurality of single cells is randomly placing into the plurality of compartments, such that the average number of cells per compartment, λ₂, is less than 1. These random placement steps may be performed in any order. Nucleic acid is then released from each single cell and captured via the capture moiety, such that nucleic acid from each single cell is tagged with a unique identification sequence.

### CROSS REFERENCE TO RELATED APPLICATIONS

This application is a continuation of U.S. application Ser. No. 15/025,874, filed on Mar. 29, 2016, which is a US National Phase of PCT Application No. PCT/EP2014/070824, filed on Sep. 29, 2014, which claims priority to GB Application No. 1317301.8, filed on Sep. 30, 2013, all of which are incorporated herein by reference in their entireties for all purposes.

### SEQUENCE LISTING

The instant application contains a Sequence Listing which has been submitted electronically in ASCII format and is hereby incorporated by reference in its entirety. Said ASCII copy, created on Apr. 13, 2021, is named FLUDP024C1US_SL.txt and is 5,149 bytes in size.

### FIELD

The present invention relates to a method for capturing and encoding nucleic acid from a plurality of single cells. This generates a library of encoded nucleic acids from single cells, which can then be sequenced.

### BACKGROUND

The current level of understanding of cell types, their origin, evolution and diversity is very poor, despite progress in some specific cases¹. There is no general agreement on the number of cell types in a mammalian body. For example, a recent survey found that 411 human cell types have been given names in the literature², but this number is far too low to be complete. For example, more than 60 cell types were identified in the retina, a well characterised tissue, and it seems likely that many new types of cells could be discovered if other tissues were as carefully scrutinised.

There is no agreement on what defines a cell type, and finding such a definition is an important goal of large-scale single-cell transcriptome analysis. There is also no agreed definitive list of named cell types. There is no agreement within two orders of magnitude on the number of distinct cell types present in the human body, and some scientists question whether the concept of cell type even makes sense.

As a starting point, cell types can be provisionally identified as cells whose global transcriptional states are similar. Just how similar, and just which parts of the transcriptome are relevant, will be crucial questions for the future. But this provisional concept of cell type leads to an unbiased method of cell type discovery (see FIG. 1): a large, unbiased sample of cells is collected from each tissue of interest, transcriptomes are generated for each and computational methods are used to find sets of similar cells. A sample of cells is taken from the tissue of interest, with the aim of obtaining a representative sample of the types of cells present in the tissue. Each cell is profiled using single-cell RNA-sequencing, and the resulting expression profiles are clustered. The result is a map of ‘cell space’, where similar cells are grouped close to each other. In practice it will be necessary to collect and analyse thousands of cells in each tissue, or millions of cells, to make a comprehensive cell space map of whole organism.

Established clustering and dimension-reduction methods, such as principal component analysis, K-means and hierarchical clustering, and affinity propagation are useful starting points. For example, Topological Data Analysis (e.g. using the Iris software package) may be used. This can reveal structures in cell maps that cannot be discovered by, for example, principal component analysis.

Hundreds or thousands of single-cell transcriptomes are already being analysed. However, despite advances in single-molecule sequencing³⁻⁵, it is not currently possible to sequence RNA directly from single cells. Thus, RNA needs to be converted to cDNA and amplified, and this must be achieved with minimal losses and without introducing too much quantitative bias. The ultimate goal of quantitative single-cell transcriptome analysis must be to count every RNA molecule in the cell exactly, resulting in zero technical error. The present inventors (in collaboration with Jussi Taipale) and others have demonstrated that this is possible by using unique labels for molecules⁶⁻¹⁰. After amplification and deep sequencing, each original molecule can be identified. As long as the sample is sequenced deeply enough, so that each molecular label is observed at least once, differences in amplification efficiency do not matter. The use of unique molecular labels is a key advance that will enable more quantitative analysis of single cell transcriptomes.

Another source of error is losses, which can be severe. The detection limit of published protocols is 5 to 10 molecules of mRNA, indicating that 80-90% of mRNA was lost. These losses are especially disturbing in small cells, such as stem cells, where the mRNA content is low to begin with.

The earliest single-cell transcriptomes were generated by in vitro transcription (IVT11), and recently IVT was used to produce libraries for Illumina sequencing, in a method called CEL-seq¹². The chief advantage of IVT is the linear amplification, which should in theory be less biased than exponential amplification methods such as PCR. A disadvantage is that the resulting library is biased towards the 3′ end of genes, and this bias can be difficult to control. In contrast, PCR-based protocols are capable of amplifying full-length cDNA.

The second approach is to add a homopolymer tail to the first-strand cDNA, which allows the cDNA strand to be amplified by PCR. An early example used dGTP-tailing followed by PCR¹³. Subsequently, this protocol was optimized¹⁴ and adapted for sequencing¹⁵. Like IVT, homopolymer tailing is biased towards the 3′ end.

The third approach uses ‘template switching’: reverse transcriptases of the MMLV family tend to add a short tail of (preferentially) cytosines to the end of the first-strand cDNA. If a helper oligonucleotide, carrying a short GGG motif, is included in the reaction, it will anneal to the cytosine motif and reverse transcriptase will switch template and copy the helper oligo sequence¹⁶. The result is that an arbitrary sequence can be introduced at the 5′ end (by tailing the reverse transcription primer) and at the 3′ end (by template switching) of the cDNA, allowing subsequent amplification by PCR. Two alternative approaches have been published for processing the full-length cDNA: STRT17, which isolates and sequences the 5′ end, corresponding to the transcription start site (TSS); and SMART-seq18, which fragments the cDNA and generates reads covering the full length of each transcript.

The present invention aims to develop a method with the necessary scale to approach a million single-cell transcriptomes. The method involves capturing single cell transcriptomes and encoding these transcriptomes with cell-specific barcodes.

### SUMMARY

The present inventors have developed a method for the quantitative analysis of single cell transcriptomes. This allows the transcriptomes of tens and thousands of single cells to be captured efficiently in a short time scale, enabling large-scale, unbiased cell-type discovery. The present inventors believe that cell types are characterised by distinct patterns of gene expression, which are ultimately generated by distinct patterns of transcription factor activity. The method of the invention disclosed herein will help to settle the question of cell types, as it will make it possible to perform large-scale unbiased cell-type discovery using single-cell transcriptomics.

In a first aspect, the invention provides a method for capturing and encoding nucleic acid from a plurality of single cells, wherein the method comprises:
(i) randomly placing a plurality of solid supports into a plurality of compartments, such that the average number of solid supports per compartment, λ₁, is less than 1, wherein each solid support carries (a) a unique identification sequence and (b) a capture moiety;
(ii) randomly placing a plurality of single cells into the plurality of compartments, such that the average number of cells per compartment, λ₂, is less than 1;
(iii) releasing nucleic acid from each single cell; and
(iv) capturing the nucleic acid from each single cell via the capture moiety, such that nucleic acid from each single cell is tagged with a unique identification sequence,
wherein steps (i) and (ii) may be performed in any order.

Preferably, the average number of solid supports per compartment, λ₁, and the average number of single cells per compartment, λ₂, are selected such that 2/(1+λ₁)(2+λ₂)≥90%, ≥95%, ≥96%, ≥97%, ≥98% or ≥99%.

The plurality of solid supports comprising (a) a unique identification sequence and (b) a capture moiety are preferably generated prior to step (ii) by emulsion PCR or split-and-pool combinatorial synthesis.

The plurality of compartments may, for example, be wells of a microwell array, or be droplets formed by an emulsifying or droplet microfluidics apparatus.

The volume of each compartment is preferably such that only a single solid support can fit into each compartment.

The solid support is preferably a microbead.

The unique identification sequence is preferably an oligonucleotide.

The capture moiety is preferably a nucleic acid complementary to cellular nucleic acid.

When both the unique identification sequence and the capture moiety are nucleic acid sequences, they may both be part of the same oligonucleotide.

Each solid support may carry a plurality of different capture moieties. When the unique identification sequence and the capture moiety are both nucleic acid sequences and are part of the same oligonucleotide, the solid support may carry a plurality of oligonucleotides, each comprising a unique identification sequence and a different capture moiety.

The nucleic acid to be captured and encoded is preferably RNA, such as messenger RNA (mRNA), ribosomal RNA (rRNA), transfer RNA (tRNA), non-coding RNAs (ncRNAs), mitochondrial RNA; nuclear or mitochondrial DNA; or microbial or viral RNA or DNA.

After step (iv), the method may optionally include the step of synthesising cDNA from the captured nucleic acid. cDNA may be further processed using any of a variety of well-known methods, to prepare for analysis by DNA sequencing. This may include selecting particular target sequences using targeted enrichment methods based on hybridization, ligation and/or PCR. It may also include steps such as synthesizing a second strand cDNA, amplification, fragmentation, adapter ligation and/or size selection.

These and other aspects of the invention are described in further detail below.

### BRIEF DESCRIPTION OF DRAWINGS

FIG. 1 is a schematic of cell type discovery by unbiased sampling and transcriptome profiling of single cells. (a) An unbiased sample of single cells is obtained. (b) Single cell expression profiles are generated. (c) Cell types are identified by clustering.

FIG. 2 is a schematic showing transcriptome capture and encoding.

FIG. 3 shows a microfluidic device. The inset (which is an actual micrograph of the junction) illustrates how the inputs do not mix prior to the junction, due to laminar flow, and how droplets are formed with half their contents from each input liquid.

FIG. 4 is a schematic showing the split-and-pool combinatorial synthesis strategy for making encoded beads.

FIG. 5 is a schematic showing the emulsion PCR synthesis strategy for making encoded beads.

FIG. 6 illustrates a design for building a library of encoded beads using split-and-pool analysis.

FIG. 7 shows an example strategy for using encoded beads to analyze RNA in single cells. Encoded beads carry oligonucleotide primers having a bead-specific identifying sequence (“CellID”) and a target-specific capture sequence (“Tsp1”). The target-specific primer directs first-strand synthesis of a complementary DNA (cDNA), shown as a dashed line. Subsequently, a reverse primer (“Tsp2”) directs synthesis of a second strand, resulting in a product suitable for sequencing. The final product includes adapter sequences (P1, P2A and P2B), an insert (middle, wide line) and an identifying sequence (CellID).

FIG. 8 shows how the probability of obtaining a high proportion of single cells on single beads depends on the bead and cell concentrations in terms of the average number of beads or cells per droplet.

FIG. 9 shows a cell map of the dorsal root ganglion.

FIG. 10A shows an example strategy for cDNA synthesis on encoded beads, and FIGS. 10B to 10D show the results of cDNA synthesis using this strategy (BioAnalyzer electrophoresis plots).

FIGS. 11A to 11D show the results of bioinformatics analyses of barcodes generated by split-and-pool. FIG. 11A shows the proportion of complete (3 modules) and incomplete (2 or less modules) barcodes. FIG. 11B shows the read distribution on ranked barcodes (filled area), and its first derivative (line). The minimum of the derivative around 3256 barcodes indicates the estimated number of correctly barcoded beads, and coincided with the estimated number of beads input (3200). FIG. 11C shows the cumulative number of beads assigned to ranked barcodes. FIG. 11D shows the read density as a histogram on the 3256 beads.

FIGS. 12A to 12D show scatterplots of randomly selected pairs of barcodes (i.e. beads). Each dot shows the number of reads mapped to a particular human gene.

FIGS. 13A to 13D show the design and operation of a custom PDMS microwell array. FIG. 13A shows the well geometry designed to fit a single cell and single 20 μm polystyrene bead snugly. FIG. 13B shows cells (translucent) and beads (opaque) in wells, before and after lysis. The arrowhead points to a cell that disappears after lysis. FIG. 13C shows a holder for three microwell arrays. FIG. 13D shows cDNA synthesized from single cells captured on single beads in the microwell array format.

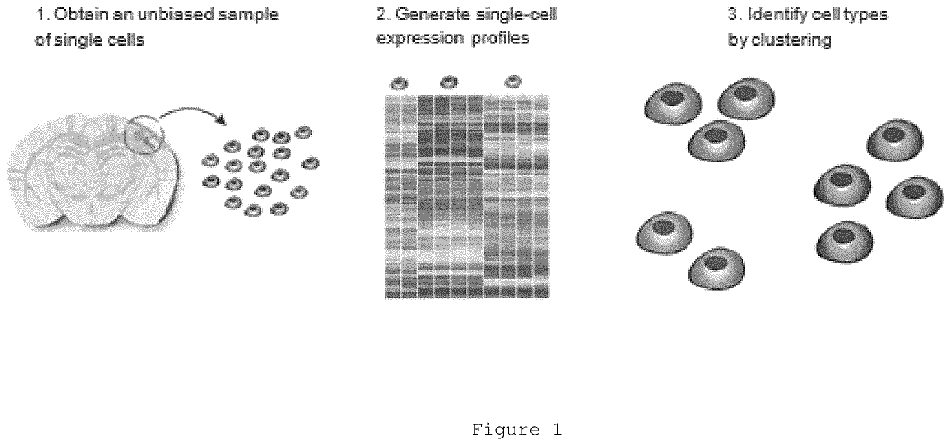

### DETAILED DESCRIPTION

The present invention relates to a method for capturing and encoding nucleic acid from a plurality of single cells.

This method allows quantitative analysis of tens of thousands of single cells in a short time frame.

The method is suitable for capturing and encoding any cellular nucleic acid, including RNA (e.g. mRNA, rRNA, tRNA, ncRNAs, mitochondrial RNA); nuclear/chromosomal or mitochondrial DNA; microbial or viral RNA or DNA.

Nucleic acid can be captured and encoded from any cell type. These cells may be any size. The size of the compartments used for capture may be adjusted to suit the target cell type. For example, bacterial cells could be analyzed in smaller compartments than mammalian cells, which are generally larger. In one embodiment, nucleic acids are captured from mammalian cells of 8-20 μm diameter encapsulated in droplets of 60-80 μm diameter.

The method is able to process thousands or millions of cells in parallel. Therefore, the plurality of single cells may be at least 10, at least 50, at least 100, at least 500, at least 1000, at least 2000, at least 3000, at least 4000, at least 5000, at least 6000, at least 7000, at least 8000, at least 9000, at least 10,000, at least 20,000, at least 30,000, at least 40,000, at least 50,000, at least 60,000, at least 70,000, at least 80,000, at least 90,000, at least 100,000, at least 200,000, at least 300,000, at least 400,000, at least 500,000, at least 600,000, at least 700,000, at least 800,000, at least 900,000, at least 1,000,000, at least 2,000,000, at least 3,000,000, at least 4,000,000, at least 5,000,000, at least 6,000,000, at least 7,000,000, at least 8,000,000, at least 9,000,000, at least 10,000,000, at least 20,000,000, at least 30,000,000, at least 40,000,000, at least 50,000,000, at least 60,000,000, at least 70,000,000, at least 80,000,000, at least 90,000,000 or at least 100,000,000 cells.

The method includes the step of randomly placing the plurality of single cells into a plurality of compartments such that the average number of cells per compartment, λ₂, is less than 1. This means that each compartment is unlikely to contain more than a single cell. Preferably, λ₂ is less than 0.9, less than 0.8, less than 0.7, less than 0.6, less than 0.5, less than 0.4, less than 0.3 or less than 0.2.

The single cells are typically provided as a suspension of dissociated single cells. This provides an unbiased sample of single cells dissociated from each tissue. The cells are preferably isolated rapidly to prevent transcriptional changes during cell preparation.

Typically, the cells are contained in a volume of 1-1000 μl isotonic buffer. Depending on the cell type, it may be desirable to add nutrients, growth factors or other such components to the buffer.

The volume of each compartment should be greater than the volume of the largest single cell to be captured. Preferably, the diameter of each compartment is from about 1 μm to about 1 mm. Therefore, the preferred diameter of each compartment will depend on the nature of the target cells. For example, when the single cells are bacterial cells, the diameter of each compartment is preferably 1-10 μm. When the single cells are typical mammalian cells, the diameter of each compartment is preferably 10-100 μm. When the single cells are large mammalian cells (such as early embryos or Purkinje cells), plant cells or protists, the diameter of each compartment is preferably 100-1000 μm.

The plurality of compartments may, for example, be wells on a microwell array. A suspension of single cells may be pipetted onto the microwell array and the cells allowed to settle into the microwells. The number of cells is adjusted so that there is a low probability of having more than one cell per well, i.e. the average number of cells per compartment is less than one. This means that each well is unlikely to contain more than a single cell. Therefore, some wells will be empty. The cells may settle into the wells by gravity flow, or may be forced by centrifugation. The microfluidic chip may comprise a glass bottom layer suitable for microscope imaging, a silicon layer having etched microwells and/or a plastic enclosure or lid having an inlet and an outlet to allow easy addition and removal of reagents on the microwell array. The thickness of each layer is arbitrary and may be adjusted to fit manufacturing or imaging constraints. The enclosure or lid is not required, but may be eliminated or replaced with a jig or other contraption intended to facilitate liquid flow across the microwell array. Likewise, the bottom layer need not be transparent if the intended application does not require imaging of the captured cells. The well diameters may vary across the full range of cell sizes, from about 1 μm to about 1 mm.

In another embodiment, the plurality of compartments may be droplets formed by an emulsifying or droplet microfluidics apparatus. In this embodiment, an aqueous input is used to make droplets using an oil carrier in a droplet microfluidic chip. This means that the number of cells that are processed can be easily adjusted by providing a larger or smaller volume of input cells. A means for controlling flow in a droplet microfluidic device is typically required, which may for example be based on a controlled pressure pump or a syringe pump.

The method also includes the step of randomly placing the plurality of solid supports into a plurality of compartments such that the average number of solid supports per compartment, λ₁, is less than 1. This means that each compartment is unlikely to contain more than a single solid support. Preferably, λ₁ is less than 0.9, less than 0.8, less than 0.7, less than 0.6, less than 0.5, less than 0.4, less than 0.3 or less than 0.2.

As disclosed above, the plurality of compartments may, for example, be wells on a microwell array. In this embodiment, a solution containing a plurality of solid supports is typically flowed over the microwell array. Due to the geometry of the wells (width and aspect ratio), cells do not escape from the wells. The plurality of solid supports are typically added at a density designed to place at most one solid support per well. Solid supports are allowed to settle in the microwells, and will reside above cells in those wells that contain single cells, thereby trapping the cells. The diameter of the solid supports is typically adjusted to prevent passage of cells between the solid support and the well wall. Optionally, the well depth may be adjusted to prevent loading more than one solid support per well; this can allow very high occupancy of the wells without risk of doublets, i.e. two solid supports in a single well.

Also as described above, in another embodiment, the plurality of compartments may be droplets formed by an emulsifying or droplet microfluidics apparatus. In this embodiment, an aqueous input containing a plurality of solid supports is used to make droplets using an oil carrier in a droplet microfluidic chip. This means that the number of solid supports can be easily adjusted by providing a larger or smaller volume of input solid supports. A means for controlling flow in a droplet microfluidic device is typically required, which may for example be based on a controlled pressure pump or a syringe pump.

Each solid support carries (a) a unique identification sequence and (b) a capture moiety. Each unique identification sequence is different for each compartment, such that each compartment carries a unique identification motif. The unique identification sequence provides a cell-specific identifying sequence, or barcode, and is preferably an oligonucleotide. In this embodiment, i.e. when the unique identification sequence is an oligonucleotide, the oligonucleotide is also known as an “encoding primer”. This enables the targeted nucleic acid from a single cell to be encoded, or tagged, with a unique identifying sequence. The identifying sequence may be varied within a set of encoding primers such that each compartment carries a unique identifying motif. This motif need not be a single sequence, but can comprise a family of sequences, for example by the use of degenerate or unspecified bases, provided each individual sequence in the family of sequences can be uniquely identified with a single encoding primer species. The oligonucleotide may include natural nucleotides, such as DNA or RNA nucleotides, as well as modified nucleotides and other modifying moieties such as dyes, functional groups (e.g. amines or biotin) or spacers. The unique identification sequence may also include one or more sample barcodes, primer annealing motifs, spacers and/or cleavable moieties.

The encoding primers may be designed to include the necessary adapter sequences for sequencing, e.g. Illumina or Complete Genomics sequencing. Upon reverse transcription and circularisation, a cDNA product is formed which is ready to sequence on either platform without amplification. The method of the invention is likely to be able to capture nucleic acid (e.g. cDNA) from approximately 10,000 cells, or more, generating approximately 3 billion encoded nucleic acid molecules (e.g. encoded cDNA molecules). This is enough to fill a whole Illumina flowcell (given 50% loss), or a single lane on Complete Genomics. Since the whole process takes place on solid supports (e.g. beads), the final product can be released directly into a suitable sequencing buffer without losses and will already be single-stranded.

The capture moiety can be any reactive or affinity reagent that allows the identifying sequence of the unique identifying sequence to become physically linked to the target nucleic acid. The capture moiety is preferably a nucleic acid complementary to the desired target nucleic acid population (i.e. cellular nucleic acid). Suitable capture moiety nucleic acid sequences include oligo-dT (to capture polyadenylated mRNA), random hexamers (or longer random motifs; to capture total RNA) or gene-specific sequences. Each solid support may carry a collection of capture moiety nucleic acid sequences (for example, multiple gene-specific sequences). Capture moiety nucleic acid sequences can contain modified nucleotides such as locked nucleic acids (LNA) to improve capture efficiency. When both the unique identification sequence and the capture moiety are nucleic acids, they may both be part of the same oligonucleotide or encoding primer. For example, the encoding primer may be a polynucleotide comprising an identifying sequence and a capture moiety designed to capture a desired nucleic acid fraction. In this embodiment, the encoding primer may, for example, include an oligo-dT sequence, a random primer, one or more gene-specific primers or an affinity reagent.

Alternatively, the capture moiety may be an affinity reagent, for example an antibody. In this approach, the capture moiety may bind a moiety attached to the target nucleic acid, such as a bound protein or a modified nucleotide. Upon binding, the target nucleic acid becomes linked with the unique identification sequence (e.g. the encoding primer), but not covalently. A covalent bond can be optionally formed using a subsequent enzymatic step, for example a DNA or RNA ligation, which will preferentially join nucleic acids that are held in close proximity.

Each solid support may carry a plurality of different capture moieties. Each different capture moiety may recognise a different target in each single cell. This means that the method of the invention may be used to analyse multiple targets in each single cell. This is known as multiplex targeting. Preferably, the unique identification sequence and the capture moiety are both nucleic acid sequences and are part of the same oligonucleotide or encoding primer. In this case, the encoding primer is also known as a “target-specific encoding primer”. The solid support may carry a plurality of oligonucleotides or target-specific encoding primers, each comprising a unique identification sequence and a different capture moiety. Preferably, the solid support is a microbead carrying a plurality of different target-specific encoding primers, each comprising a different capture moiety and unique identification sequence. This means that the method of the invention can be used to capture multiple distinct target nucleic acids from each single cell.

The plurality of solid supports are preferably microbeads. Such microbeads are well known in the art and are commonly used for purification of nucleic acids or proteins, and for performing enzymatic or chemical reaction on an immobilized substrate. Microbeads are typically made of a polymer such as polystyrene and can be paramagnetic to allow simple repeated purification using magnetic immobilization of the beads. The bead surface can be modified to obtain desired physico-chemical properties such as hydrophilicity, and to make the surface reactive to a target molecule.

In one embodiment, the unique identification sequence is immobilised on a microbead. When the unique identification sequence is an oligonucleotide, it may be referred to as an encoding primer and the microbead may be referred to as an “encoding bead”. A plurality of encoding beads may be generated, each bead carrying a unique encoding primer.

The size of microbeads affects the surface area and thereby the number of encoding primers that can be placed on each bead. For example, it is estimated that 20 μm streptavidin-coated polystyrene beads can carry about 100 million encoding primers, of which about 10 million can simultaneously capture target RNA without steric hindrance. This is more than sufficient for capturing the mRNA of typical mammalian cells (0.1-1 million molecules) and is almost sufficient for capturing total RNA from single mammalian cells (3-30 million molecules). However, by scaling bead size and thus surface area, any desired binding capacity can be obtained. Since surface scales with the square of the diameter, a 40 μm bead would bind up to 400 million encoding primers.

Typically, it is desirable to produce a large number of microbeads carrying distinct encoding primers. It is trivial to produce hundreds, thousands or tens of thousands of distinct encoding primers simply by individual oligonucleotide synthesis, either directly on the beads, or separately followed by bead immobilisation. Distinct sequences are used and immobilised on distinct populations of microbeads. Beads are then pooled, resulting in a population of beads carrying a large number of bead-specific encoding primers.

However, if the desired number of distinct encoding primers required exceeds manufacturing capacity (or the cost becomes prohibitive), microbeads can instead be produced by compartmentalized PCR, for example emulsion PCR, droplet PCR or picotiter-plate PCR. In this approach, beads are mixed with encoding primers and compartmentalised PCR is performed in such a way that typically a single encoding primer oligonucleotide is amplified onto each bead. If the input Encoding Primer contains a degenerate sequence of, say, 20 nucleotides, there will be the possibility of producing more than one trillion distinct encoded microbeads.

The plurality of solid supports may be generated by:

(a) randomly placing a plurality of solid supports into a plurality of compartments, such that the average number of solid supports in each compartment is less than 1;
(b) randomly placing a plurality of encoding primers comprising a unique identification sequence and a capture moiety into the plurality of compartments, such that the average number of encoding primers in each compartment is less than 1;
(c) mixing the plurality of solid supports and the plurality of encoding primers, and causing the encoding primers to be amplified, to generate encoded solid supports;
wherein steps (a) and (b) can be performed in any order and wherein following step (c), the encoded solid supports can be used in step (ii) of the method of the invention.

The steps of (i) randomly placing the plurality of single cells into the plurality of compartments and (ii) randomly placing the plurality of solid supports into the plurality of compartments may be performed in any order.

The volume of each compartment is preferably such that only a single solid support can fit into each compartment. Alternatively, the size of the solid support may be adjusted such that only a single solid support can fit into each compartment. This helps to prevent more than one solid support being placed into each compartment.

Once a cell and a solid support comprising a unique identification sequence and a capture moiety have been placed together in a compartment, nucleic acid is released from each single cell. This may be achieved by lysing the cell under conditions that promote annealing of the target nucleic acid to the capturing moiety of the unique identification sequence. Cells are preferably lysed rapidly to prevent transcriptional changes during cell preparation. Depending on the design of the compartment, the lysis and capturing steps may be simultaneous, or may be performed by sequential addition of suitable reagents.

Additional, optional steps may be included, such as washing the cells, denaturing the target nucleic acid and similar operations.

For example, when the plurality of compartments are wells on a microwell array, nucleic acid is released from each single cell by flowing lysis buffer over the microwell array and allowing it to diffuse down into the microwells. The lysis buffer is designed to lyse the cells while also promoting RNA capture. Many such buffers are known in the art (Sambrook et al., “Molecular Cloning: A Laboratory Manual), but generally they contain salt, detergent and a buffering agent. For example, an efficient lysis buffer contains 500 mM LiCl, 100 mM Tris-Cl pH 7.5, 1% lithium dodecyl sulfate, 10 mM EDTA and 5 mM DTT.

Once the nucleic acid has been released from each single cell, it is then captured via the capture moiety, such that nucleic acid from each single cell is tagged with a unique identification sequence to generate an encoded nucleic acid, e.g. encoded DNA. For example, when the plurality of compartments are wells on a microwell array, once a cell lyses, its RNA spills out and begins diffusing away. As it diffuses, it must pass the capture moiety linked to the solid support (e.g. an encoded bead) loaded on top of the cell, and the targeted RNA fraction is captured in passing.

The generation of encoded nucleic acid results in the formation a library of nucleic acid molecules derived from the nucleic acids of one or more cells, each molecule carrying a sequence tag that identifies its cell of origin. The resulting nucleic acid library may be amenable to sequencing on any modern DNA sequencing platform.

Once the target nucleic acids have been captured by the capture moiety, the target nucleic acid is linked to the compartment specific identifying sequence. At this point, further processing may proceed in the separate compartments, or the contents of each compartment may be pooled in a single reaction vessel.

Finally, the solid supports (e.g. microbeads) are unloaded and collected in a single reaction tube. Unloading can be by pipetting, by magnetic force or by centrifugation.

Collected beads are washed and post-processed as a single reaction. Post-processing steps are application specific (see examples, below).

There are many possible variations of the described embodiments, provided single cells are captured in compartments which are also made to contain unique identification sequences, e.g. encoding primers. Embodiments of the invention will differ according to how the compartments are formed, e.g. emulsions, droplets, microfluidic chambers, microwell arrays, how the unique identification sequences (e.g. encoding primers) are brought to the chambers (e.g. carried on microbeads, immobilised on a reaction chamber surface, injected by microfluidic valves and ports), what reaction steps are performed in the compartments (e.g. just the nucleic acid capture step, or also reverse transcription, or also optionally some or all of the post-processing steps).

In order to carry out the method as efficiently as possible, it is desirable for a high proportion of the compartments to contain a single cell and a single solid support carrying a unique identification sequence and a capture moiety.

Using microbeads as exemplary solid supports, the table below lists (for different cell and microbead concentrations) the expected fraction of microbeads that carry RNA from single cells, from double cells and from split cells. Empty microbeads are not counted. The last two columns indicate the number of droplets and microbeads needed to generate about 10K single cells.

TABLE 1
yields of single cells on single microbeads
as a function of input concentrations.

| Micro- | | | | | # | | Drop- | |
|---|---|---|---|---|---|---|---|---|
| bead | Cell | Single | Double | Split | Drop- | # | let | |
| conc. | conc. | cells | cells | cells | lets | Beads | Volume | |
| 10% | 10% | 86% | 4% | 9% | 1M | 100K | 250 | μL |
| 10% | 2% | 90% | 0.9% | 9% | 5M | 500K | 1.25 | mL |
| 10% | 1% | 90% | 0.5% | 9% | 10M | 1M | 2.5 | mL |
| 2% | 2% | 97% | 1% | 2% | 25M | 500K | 6.25 | mL |
| 1% | 1% | 99% | 0.5% | 1% | 100M | 1M | 25 | mL |

The table was derived as follows. If objects are distributed randomly among compartments of identical size, the number of objects (e.g. microbeads, cells or template molecules) found in each compartment (e.g. droplet, microwell) follows the Poisson distribution. If the concentration of objects is C and the volume of each compartment is V, then the expected average number of objects per compartments is λ=C/V and the probability of finding exactly x objects in a compartment is given by

$$P ( x \text{\textbar} λ ) = \frac{{λ}^{x} {e}^{- λ}}{x !}$$

which is the Poisson distribution.

C can be controlled by changing the concentration of objects, and V can be controlled within a large range by varying the size of the compartments (e.g. by making different-size wells or by adjusting the relative flow rates of oil and water to make different-sized droplets). Thus, λ can be controlled nearly arbitrarily, and in particular, it can be arbitrarily made smaller than one by simply diluting the objects.

When mixing two types of objects (with expected average number of objects per compartments λ₁ and λ₂), the probability of getting exactly x and y objects in a compartment is the product of the individual probabilities:

$$P ( x , y \text{\textbar} {λ}_{1} {λ}_{2} ) = P ( x \text{\textbar} {λ}_{1} ) P ( y \text{\textbar} {λ}_{2} ) = \frac{{λ}_{1}^{x} {e}^{- {λ}_{1}}}{x !} \frac{{λ}_{2}^{y} {e}^{- {λ}_{2}}}{y !}$$

Using these formulas, the probabilities of the most interesting cases can be calculated¹, i.e. when there are zero, one or two objects per compartment: ¹ Mathematica code to produce the table: Table[FullSimplify[PDF[PoissonDistribution[Subscript[\[Lambda], 1]], x]PDF[PoissonDistribution[Subscript[\[Lambda], 2]], y], {x>=0, y>=0}]/.{x->a, y->b}, {a, 0, 2}, {b, 0, 2}]//Transpose//TableForm

TABLE 2
Probabilities of obtaining compartments with zero, one or two objects.

| | x = 0 | x = 1 | x = 2 |
|---|---|---|---|
| y = 0 | e^{-λ}^{₁}^{-λ}^{₂} | e^{-λ}^{₁}^{-λ}^{₂}λ₁ | $\frac{1}{2} {e}^{- {λ}_{1} - {λ}_{2}} {λ}_{1}^{2}$ |
| y = 1 | e^{-λ}^{₁}^{-λ}^{₂}λ₂ | e^{-λ}^{₁}^{-λ}^{₂}λ₁λ₂ | $\frac{1}{2} {e}^{- {λ}_{1} - {λ}_{2}} {λ}_{1}^{2} {λ}_{2}$ |
| y = 2 | $\frac{1}{2} {e}^{- {λ}_{1} - {λ}_{2}} {λ}_{2}^{2}$ | $\frac{1}{2} {e}^{- {λ}_{1} - {λ}_{2}} {λ}_{1} {λ}_{2}^{2}$ | $\frac{1}{4} {e}^{- {λ}_{1} - {λ}_{2}} {λ}_{1}^{2} {λ}_{2}^{2}$ |

In the method of the invention, low concentrations of beads and cells are used, so that the probability of getting more than two objects per compartment is negligible.

Table 2 gives the probability of finding each type of compartment. Usually, the objects are of more interest. After combining beads and cells in droplets, beads are collected and the probabilities, per bead, that this bead was part of a droplet that contained one or two beads as well as one or two templates are calculated. This requires two modifications to the table. First, (arbitrarily letting x represent beads), the column for x=0 is removed, where there are no beads, and the row for y=0, where there are no cells. Second, the column for x=2 is multiplied by two, since each compartment then contains two beads, and hence the probability of this event per bead is twice as large. Normalising by the total probability, the following formulae² are obtained: ² Mathematica code to generate this table: table=Table[FullSimplify[PDF[PoissonDistribution[Subscript[λ,1]],x]xPDF[PoissonDistribution[Subscript[λ,2]],y],[x is 0 y is 0]]/.{x->a,y->b},{a, 1, 2},{b, 1, 2}]//Transpose, const=Total[Total[table]], table/const//FullSimplify//TableForm

TABLE 3
Probabilities of finding one or two beads in a single compartment.

| | x = 1 | x = 2 |
|---|---|---|
| y = 1 | $\frac{2}{( 1 + {λ}_{1} ) ( 2 + {λ}_{2} )}$ | $\frac{2 {λ}_{1}}{( 1 + {λ}_{1} ) ( 2 + {λ}_{2} )}$ |
| y = 2 | $\frac{{λ}_{2}}{( 1 + {λ}_{1} ) ( 2 + {λ}_{2} )}$ | $\frac{{λ}_{1} {λ}_{2}}{( 1 + {λ}_{1} ) ( 2 + {λ}_{2} )}$ |

These formulae were used to calculate Table 1 above, with the following interpretations:

TABLE 4
Interpretation of cases shown in Table 1.

| Case | Interpretation |
|---|---|
| x = 1, y = 1 | Single cells |
| | (fraction of cell-carrying beads that carry a single cell) |
| x = 1, y = 2 | Double cells |
| | (two cells on the same bead) |
| x = 2, y = 1 | Split cells |
| | (one cell on two different beads) |
| x = 2, y = 2 | Split and double |

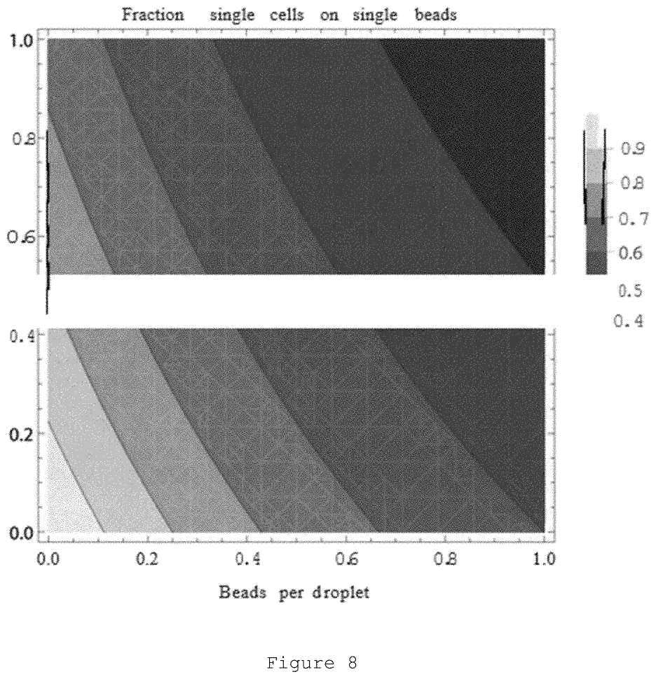

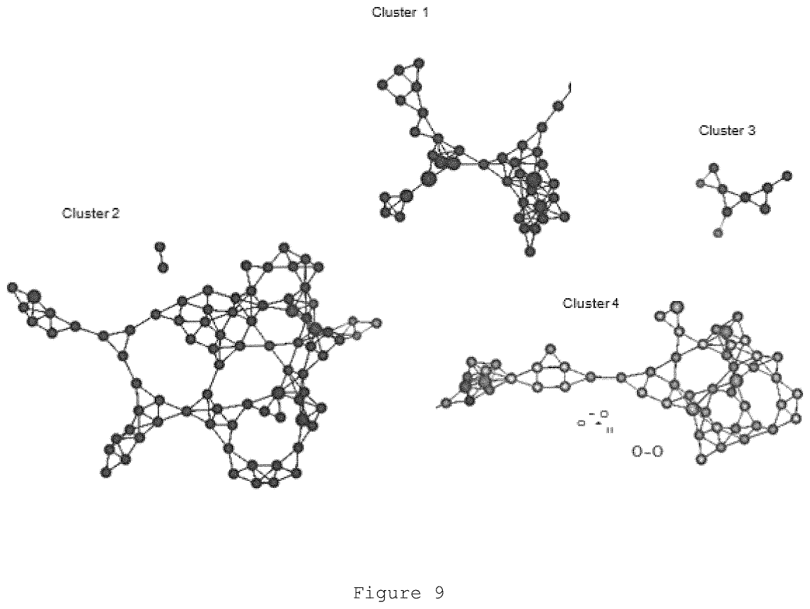

FIG. 8 shows how the probability of the good outcome, i.e. a high probability of obtaining a high proportion of compartments containing a single cell and a single bead.

The probability of obtaining a good outcome can be increased arbitrarily by diluting all objects as needed. However, this results in a smaller fraction of beads used, and of cells captured. For some applications (e.g. when the cells are few and precious) it may be desirable to allow a somewhat smaller fraction of single cells on single barcodes, in order to ensure capture of a larger fraction of the cells. For other applications, e.g. when the goal is to detect a rare cell type, it may be much more important to avoid generating spurious signals from mixed cells. Thus the formula and tables above can guide the user in finding the best tradeoff among the parameters, for any given application.

For example, the number of solid supports and the number of single cells to be placed into the plurality of compartments may be selected so that the probability of obtaining a single solid support and a single cell together in a single compartment is ≥50%, ≥60%, ≥70%, ≥80%, ≥90%, ≥95%, ≥96%, ≥97%, ≥98% or ≥99%. Using the formulae provided in Table 3, this means that the average number of solid supports per compartment, λ₁, and the average number of single cells per compartment, λ₂, may be selected such that 2/(1+λ₁) (2+λ₂) ≥50%, ≥60%, ≥70%, ≥80%, ≥90%, ≥95%, ≥96%, ≥97%, ≥98% or ≥99%.

Alternatively, the starting concentration of the plurality of solid supports, C₁, the starting concentration of the plurality of single cells, C₂, and the volume of each compartment, V, may be selected so that the probability of obtaining a single cell and a single solid support together in a single compartment is ≥50%, ≥60%, ≥70%, ≥80%, ≥90%, ≥95%, ≥96%, ≥97%, ≥98% or ≥99%. Using the formulae provided in Table 3 along with the fact that λ=C/V, this means that C₁, C₂, and V may be selected such that 2/[(1+C₁/V) (2+C₂/V)] is ≥50%, ≥60%, ≥70%, ≥80%, ≥90%, ≥95%, ≥96%, ≥97%, ≥98% or ≥99%.

The nucleic acid released from each single cell may then be sequenced such that the expression profile of each single cell may be determined. Clustering algorithms may be used to find sets of highly similar cells. These cells are the likely candidates for being distinct, stable cell types.

The method of the invention allows the quantitative analysis of single cell transcriptomes. The transcriptomes of tens and thousands of single cells may be captured simultaneously in a process taking less than half an hour. Each transcriptome is captured on a custom-made solid support (e.g. a microbead) and encoded with a cell-specific barcode present on each solid support. After pooling all the solid supports and single cells, reverse transcription and circularisation with a platform-specific adapter, the resulting nucleic acid (e.g. cDNA) is ready to sequence without amplification on, for example, Illumina or Complete Genomics platforms. As amplification can be avoided, the data are expected to be highly quantitatively accurate. On the Illumina platform, it would be expected that ten thousand cells per flowcell could be sequenced at a more than ten-fold reduction in cost over the present methods. On the Complete Genomics platform, it would be feasible to sequence a million single cells, achieving an almost hundred-fold reduction in cost.

One major application for single-cell transcriptomics is in the analysis of rare cell types. For example, circulating tumor cells (CTCs) can be obtained from patient blood, where typically only a handful of cells are isolated per blood sample. In many cases, the small number of CTCs will be contaminated by a larger number of normal cells, but single-cell RNA-seq could be used to differentiate between them and simultaneously obtain expression data from the tumor.

Similarly, the early human embryo by definition contains only rare cell types, which exist only transiently, yet these cells are among the most crucial in the life of any human. To understand embryogenesis, the very first cellular differentiation event (occurring some time prior to the formation of the blastocyst) is a prime model system. Furthermore, the pre-implantation embryo holds the key to many questions about fertility, regenerative capacity and the ground state of human development. These questions could be addressed using transcriptomics, by studying the cascade of events that lead to degradation of the maternal transcriptome, the emergence of the fetal transcriptome, and the interplay between maternal and paternal chromosomes. In this context, transcriptomics has the advantage of being able to use sequence polymorphisms (e.g. SNPs) to distinguish transcripts derived from each of the two parental genomes.

Another area that will benefit immensely from single-cell transcriptomics is the study of adult stem cells. These are often rare, quiescent cells, which are capable of regenerating adult tissues. In many cases, stem cells have been shown to exist in multiple states, serving distinct long- and short-term regenerative needs for the organism. Such systems consisting of stem cells, transient cell types and post mitotic differentiated cells are difficult to study, as distinct cell types are intermingled. But with single-cell RNA-seq, each cell type can be extensively sampled simply by taking unbiased samples of cells out of the tissue.

A further application area for single-cell transcriptomics is the characterization of transcriptional fluctuations. The cellular state, including its transcriptome, is in constant flux. Dynamic changes in RNA content are associated with cyclic processes, such as the cell cycle in dividing cells and the circadian rhythm. Other fluctuations are stochastic and reflect the fact that transcription is a discrete process composed of many probabilistic steps. Further heterogeneity is introduced by uneven partitioning of the cellular content at cell division. For example, unequal partitioning of mitochondria contributes to cell-to-cell differences in energy metabolism, leading to differences in ATP concentration and ultimately to global differences in transcription rate¹⁹.

Direct transcriptome analysis of large numbers of single cells should open up the study of oscillatory and stochastic regulatory processes in unperturbed cell populations. In a population of putatively identical cells, sets of co-regulated genes can be identified. Each set must be part of a functional process, such as an oscillator or a stochastic process. For example, genes that share a common upstream regulator would presumably show correlated expression. At present, the number of single cells that must be analysed in order to discover covariant genes is unknown, and finding first estimates of these numbers will be a key task in the near future.

Furthermore, there is evidence that transcription is subject to strong intrinsic fluctuations^{20, 21}. A plausible model to explain this intrinsic noise is the two-state model: each promoter flips stochastically between an active state and a silenced state. If the active state has a short duration, then transcription will occur in short bursts, leading to a rapid accumulation of mRNA, followed by a period of mRNA decay. This model leads to a prediction about the shape of the mRNA copy number distribution which can be tested against experimentally measured distributions²⁰. It is important to realise that any model of transcription that leads to a prediction about mRNA distributions (as opposed to the population mean) cannot be tested using bulk measurements, which do not give any information about the variance or any higher moments. Nonetheless, single-cell transcriptome analysis provides only a snapshot in time, and it will remain important to complement this view with dynamic long-term measurements by, e.g. time-lapse microscopy²².

### EXAMPLES

### Example 1—Production of Encoded cDNA from Single Cells

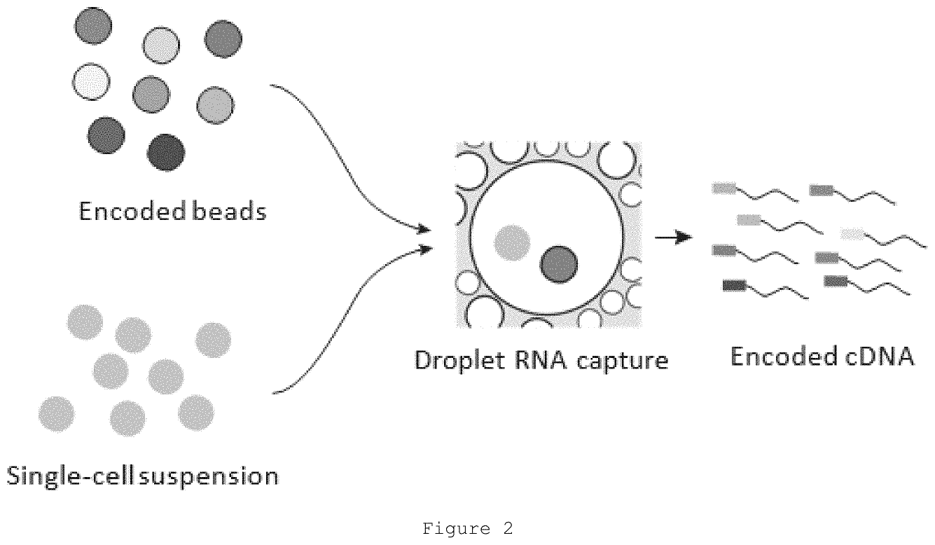

This example describes the production of encoded cDNA from single cells. The method involves (i) capturing single cell transcriptomes in a microfluidic device and (ii) encoding those transcriptomes with cell-specific barcodes. The production of encoded cDNA using a droplet-based reaction RNA capture step is illustrated in FIG. 2. This method involves two reaction stages.

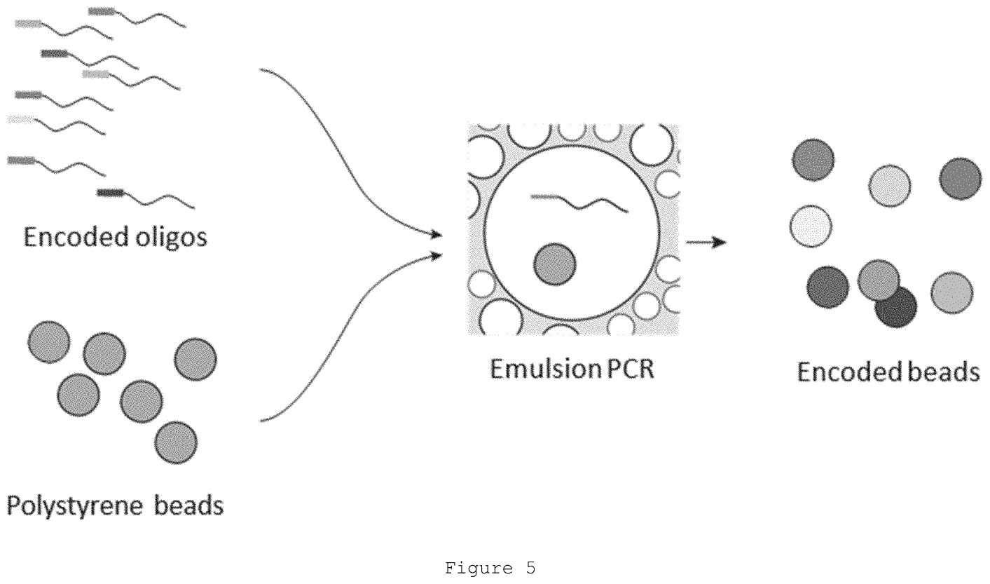

The first stage uses either split-and-pool or emulsion PCR (emPCR) to generate encoded beads, illustrated in FIGS. 4 and 5. Custom-manufactured magnetic beads (20 μm diameter polystyrene paramagnetic beads) carrying approximately 10 million oligonucleotides on their surface are used.

The second stage uses emulsion or microwell RNA capture followed by cDNA synthesis to generate encoded DNA. The result is to convert the mRNA content of thousands of single cells to encoded cDNA carrying cell-specific identifying sequences. At this stage, a stream of single cells is merged with a stream of encoded beads such that most droplets contain a single bead and a single cell (Poisson statistics). The process is illustrated in FIG. 2.

A microfluidic droplet generator is used to produce 250 pL droplets that encapsulate two input liquids. The result is to transfer the mRNA content of single cells onto beads carrying bead-specific identifying sequences. Tens of thousands of cells can be processed in millions of droplets, which minimises the risk that more than one cell (or more than one bead) is present in each droplet.

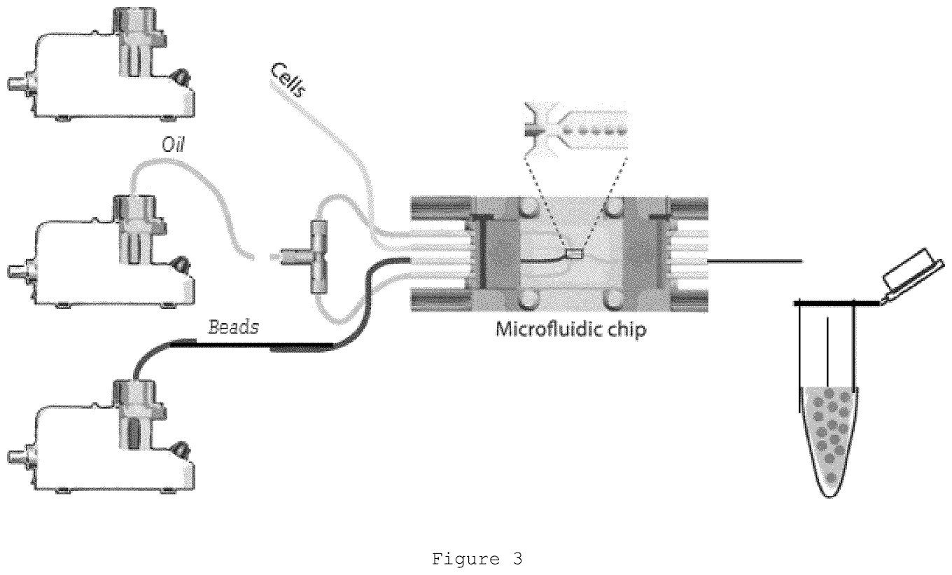

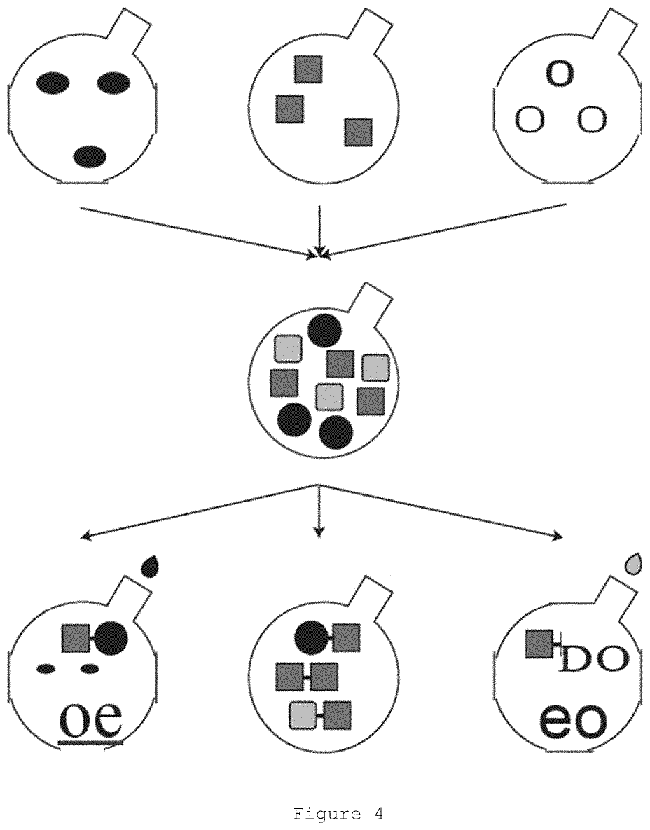

The present inventors have designed a custom droplet microfluidic device (Dolomite Microfluidics), which is capable of generating about 100 million droplets in half an hour, merging two input streams (see FIG. 3). Three closed-loop pressure-driven pumps push three input liquids into a microfluidic chip. Oil is introduced from the sides and forms the carrier phase. Two aqueous inputs (cells and beads) are introduced in parallel and merge just prior to the junction. The inset (which is an actual micrograph of the junction) illustrates how the inputs do not mix prior to the junction, due to laminar flow, and how droplets are formed with half their contents from each input liquid. After collection of the droplets, the two input liquids mix by diffusion. As the bead solution contains lysis reagents, the cells lyse, their mRNA spills out and is captured on the beads.

The droplet generator is a dual-input droplet microfluidic device generating 250 pL droplets. Aqueous streams carrying beads in lysis buffer and cells are brought together just before an X-junction, where pressure from an oil phase is used to generate a monodisperse stream of droplets, each containing an equal mixture of the two input reagents. This causes the cells to lyse and mRNA to be captured on the beads (see FIG. 3).

A. Preparing Encoded Beads by Split-and-Pool

A1. Background

Split-and-pool is a combinatorial synthesis strategy (see FIG. 4) where a combinatorial library of molecules is built by sequential steps of (1) splitting the reaction mix in multiple vessels; (2) adding to each vessel a different monomer, which is ligated to the molecules present in the vessel; (3) pooling the content of all vessels; (4) repeating this process N times to generate a pooled combinatorial library of polymers.

Split-and-pool is used to build a library of encoded beads by sequential ligation of short DNA building blocks (the monomers; a total of 192 different, 8 bp long). Thus, each bead will carry a specific sequence of building blocks. With three rounds of split-and-pool, a total of 192\hat{ }3=7 million different kinds of beads are produced, each carrying a distinct sequence of building blocks. A key benefit of split-and-pool compared with emulsion PCR is that every bead is productive. That is, every bead gets a specific sequence, and no beads get mixed sequences. In contrast, with emulsion PCR it is necessary to dilute beads and templates to reduce the number of beads that get two templates, but this leads to a majority (most likely >90%) empty beads, which must be discarded.

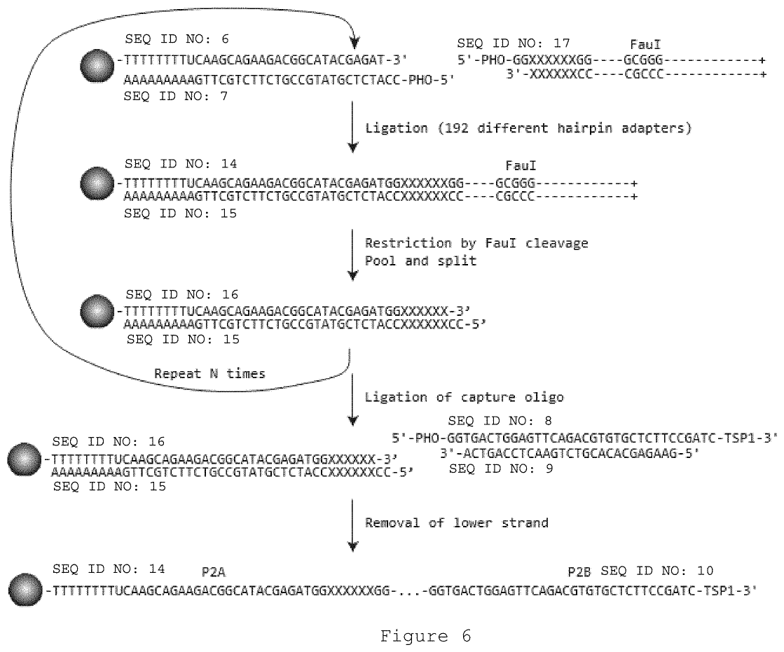

FIG. 6 illustrates a design for building a library of encoded beads using split-and-pool analysis. A hairpin adapter that can be cleaved by a restriction enzyme (FauI) in such a way that it leaves a CC overhang and a six basepair barcode sequence (selected among 192 distinct barcodes) is used. Thus, starting with a CC overhang on the beads, each round of split and pool will grow the DNA on the beads by one block and regenerate the beads for the next round. After N such rounds, synthesis is completed by ligating an adapter containing a targeting sequence (here termed TSP1, target-specific primer 1). This could be for example an oligo-dT sequence for targeting all mRNA, or a pool of gene-specific targeting sequences. Illumina adapter sequences (here termed P2A and P2B) are used so that the barcodes can be sequenced separately from the insert.

The result is a pool of beads, each carrying (1) Illumina P2A and P2B for sequencing; (2) a bead-specific barcode comprising a sequence of hexamer blocks (where each block is selected among 192 different blocks); and (3) a target-specific primer sequence. The target-specific primer (TSP) can potentially be a mixture of primers, in which case each bead will carry a mixture of TSPs. This can be used to analyse multiple defined targets per cell. In this case, each bead will carry a plurality of target-specific primers, comprising sets of target-specific primers directed against distinct targets.

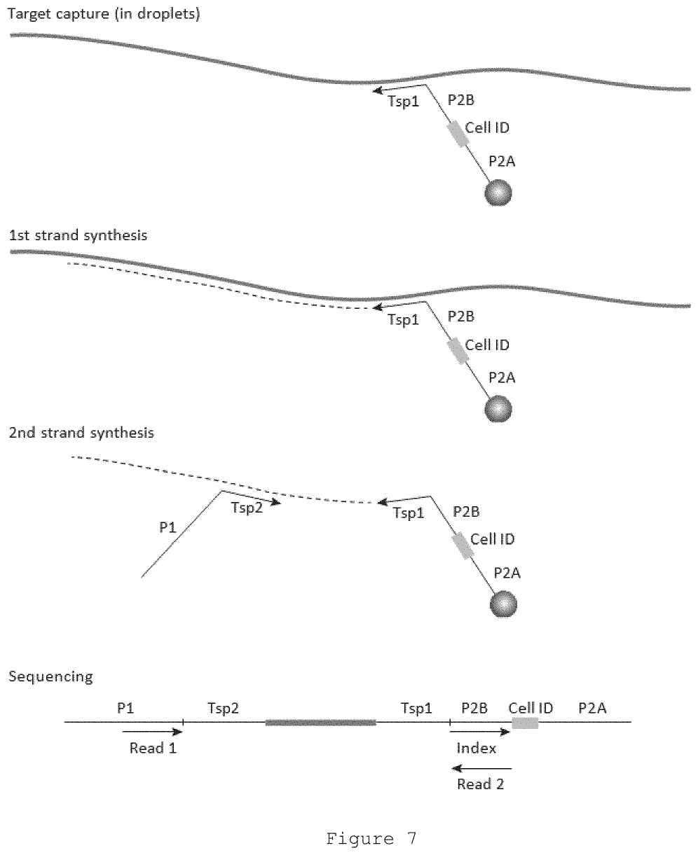

This targeting approach is illustrated schematically in FIG. 7.

A2. Preparations

N sets of splitting plates containing 12 μM hairpins in 5 μL of 1×NEB T4 Ligase Buffer are prepared. With 192 hairpins, there are two plates per set, and 2N plates in total.

Double-stranded (ds) immobilised DNA is prepared using the reagents shown in Table 5:

TABLE 5
Preparation of ds immobilised DNA.

| Reagent | Volume | Final conc. |
|---|---|---|
| bio_8T_U_P2A (100 μM) | 12 μl | 12 μM |
| P2A(rc)_9A (100 μM) | 20 μl | 20 μM |
| Water | 68 μl | |
| Total volume | 100 μl | |

Beads are prepared as follows. 1 ml (˜2M) of Capture beads (Dynabeads® MyOne™ Streptavidin C1) are used. The beads are bound and resuspended in 200 μl 2×BWT (10 mM Tris-HCl (pH 7.5), 1 mM EDTA, 2 M NaCl and 0.01% (vol/vol) Tween-20). The beads are washed 2 times in 2×BWT and then separated by briefly spinning down (NOT by magnet) to get rid of magnetic debris. The washed beads are then resuspend in 100 μl 2×BWT.

The beads are coated by adding 100 μl ds immobilised DNA to 100 μl of beads, followed by incubation at room temperature for 15 minutes. The beads were then washed twice in 1×BWT.

The beads are then “split” by being resuspended (on ice and kept cold) in the reaction mix shown in Table 6.

TABLE 6
Reaction mix for “splitting” the coated beads.

| | | Final conc. |
|---|---|---|
| Reagent | Volume | (splitting plate) |
| Water | 850 μl | |
| x10 NEB T4 Ligase Buffer | 100 μl | 1x |
| T4 ligase (400,000 U/ml) | 50 μl | 10 U/μl |
| Total volume | 1000 μl | |

The reaction mix is divided into the splitting plates at 5 μl/well in two 96-well plates before being incubated at room temperature for 20 minutes and heat inactivated at 65° C. for 10 min.

The beads are immobilized using a magnet and the supernatant removed by washing the beads 3 times in 200 ul 1×BWT. The sample is resuspended in the buffer shown in Table 7. The sample is kept on ice throughout.

TABLE 7
Composition of buffer for “pooling the beads”.

| Reagent | Volume | Final [C] |
|---|---|---|
| Water | 132 μL | |
| x10 CutSmart ™ Buffer | 15 μL | 1x |
| NEB FauI (5000 U/ml) | 3 μL | 15 U in 150 μl |
| Total volume | 150 μL | |

The sample is incubated at 55° C. for 30 minutes and heat inactivated at 65° C. for 20 min. The beads are bound and the supernatant removed by washing the beads 3 times in 200 μl 1×BWT.

The procedure is repeated for as many rounds as required from “Splitting” (normally three).

The procedure is then repeated from the “splitting” stage, but ligating a target-specific primer (TSP1) adapter instead of a hairpin adapter and omitting the restriction step.

The DNA coated Dynabeads® are optionally washed in 200 μl 1×SSC. They are then resuspended in 200 μl of freshly prepared 0.15 M NaOH and incubated at room temperature for 10 minutes. The Dynabeads® coated with biotinylated strand are washed once with 100 μl 0.1M NaOH, once with 100 μl of B&W buffer and once with 100 μl TE buffer. They are then resuspend in 500 μl TE and stored at 4° C.

B. Transcriptome Capture and Encoding

The emulsifier oil mix is prepared by adding Picosurf2 (Dolomite Microfluidics) to FC-40 at 2% final concentration. The mixture is vortexed thoroughly and incubated at room temperature for 30 minutes.

The encoded bead mix is prepared by using the reagents shown in Table 8. The mix is kept on ice until use.

TABLE 8
Composition of the encoded bead mix.

| | | |
|---|---|---|
| Reagent | Volume | Final conc. |

| | | | |
|---|---|---|---|
| 5M LiCl | 62.5 | μL | 500 mM |
| 1M TrisCl pH 7.5 | 62.5 | μL | 100 mM |
| 10% Lithium dodecyl | 62.5 | μL | 1% |
| sulfate | | | |
| 0.5M EDTA | 12.5 | μL | 10 mM |
| 100 mM DTT | 31 | μL | 5 mM |
| 10K/μL Encoded Beads | 50 | μL | 100K beads |
| Nuclease-free water | 345 | μL | |
| Total volume | 626 | μL | |

Note: Less than 50 μL beads can be used, but the yield will be correspondingly reduced.

A single-cell suspension is prepared as follows. Tissue-specific protocols are used to dissect and dissociate to a single-cell suspension. Aliquots of 100,000 cells are frozen in 625 μL cell culture medium with 10% DMSO. An aliquot is thawed and kept on ice. Note: It is important to wash the cells thoroughly before freezing, to eliminate extracellular RNA. Debris is cleared by passing cells through a cell strainer. If necessary, debris is removed by a 20% Percoll step-gradient centrifugation.

An emulsification step is then carried out as follows.

The Encoded Bead mix is loaded on the A pump and the single-cell suspension on the B pump of the droplet generator. 2 mL Emulsifier Oil Mix is loaded on the C pump. The pump is used at 10/10/20 μL/min to generate 80 μm (250 pL) droplets until the reagents run out. The total volume is about 2.5 mL and takes about one hour to collect. The emulsion is collected in a single tube kept on ice.

The tube is transferred to a thermocycler and incubated at 72° C. for 15 minutes to lyse the cells and denature and fragment RNA; then 55° C. for four hours followed by 30° C. for one hour to capture target RNAs. The emulsion is chilled on ice. The bead-positive droplets are bound and the rest of the emulsion is carefully removed. The emulsion is broken and the beads are recovered using 5 mL breaking buffer.

Post-processing begins with reverse transcription. The beads are bound and resuspended the beads in the mix shown in Table 9 prepared on ice.

TABLE 9
Composition of mix used for reverse transcription.

| Reagent | Volume | Final conc. | |
|---|---|---|---|
| 200 U/μL Superscript | 1 μL | 10 | U/μL |
| II | | | |
| 5x Superscript II | 4 μL | 1x | |
| buffer | | | |
| 20 mM DTT | 2 μL | 2 | mM |
| 100 μM dNTP | 1 μL | 5 | μM |

| | | |
|---|---|---|
| 100 mM MgCl₂ | 2 μL | 10 mM (13 mM with SSII |
| | | buffer) |
| Nuclease-free water | 10 μL | |
| Total volume | 20 μL | |

The mix is incubated at 42° C. for exactly ten minutes. The incubation time can be adjusted to change the resulting average cDNA fragment length.

Unused primers are then removed as follows. The beads are resuspended in the mix shown in Table 10.

TABLE 10
Mixture used to resuspend the beads
prior to removal of unused primers.

| Reagent | Volume | Final conc. |
|---|---|---|
| 10x Exonuclease I | 1 μL | 1x |
| buffer | | |
| Nuclease-free water | 8 μL | |
| 10 U/μL Exonuclease I | 1 μL | 1 U/μL |
| Total volume | 10 μL | |

The beads are incubated in the mixture shown in Table 6 at 37° C. for 30 minutes and then heat inactivated at 80° C. for 20 minutes. The beads are washed twice in TNT buffer.

The RNA strand is removed by resuspending the beads in the mix shown in Table 11.

TABLE 11
Mixture used to resuspend the beads
prior to removal of the RNA strand.

| Reagent | Volume | Final conc. |
|---|---|---|
| 10x RNase H buffer | 1 μL | 1x |
| Nuclease-free water | 8 μL | |
| 5 U/μL RNase H | 1 μL | 1 U/μL |
| Total volume | 10 μL | |

The mixture was incubated at 37° C. for 20 minutes and heat inactivated at 65° C. for 20 minutes. The beads were washed twice in TNT buffer.

The second strand was synthesised by resuspending the beads in the mix shown in Table 12 to anneal reverse primers:

TABLE 12
Reaction mix used to anneal reverse primers.

| Reagent | Volume | Final conc. | |
|---|---|---|---|
| 5M NaCl | 1 μL | 500 | mM |
| 1M Tris HCl pH 7.5 | 1 μL | 100 | mM |
| 100 μM TSP2 primer | 1 μL | 10 | μM |
| mix | | | |
| Nuclease-free water | 7 μL | | |
| Total volume | 10 μL | | |

The mixture was incubated at 72° C. for 5 minutes and then cooled to 20° C. The beads were washed twice in TNT buffer and resuspended in the mix shown in Table 13 to extend the second strand:

TABLE 13
Reaction mix used to extend the second strand.

| | | |
|---|---|---|
| Reagent | Volume | Final conc. |

| | | | |
|---|---|---|---|
| 10x NEBuffer2 (NEB) | 1 | μL | 1x |
| Klenow (3′-5′ exo⁻) | 1 | μL | |
| BSA (100x) | 0.1 | μL | 1x |
| dNTP | | | 250 μM each |
| Nuclease-free water | 8 | μL | |
| Total volume | 10 | μL | |

The mixture was washed twice in TNT, and then the second strand was released by incubating in 0.1 M NaOH followed by neutralisation in 0.1 M HCl and Tris.

The Encoded cDNA can be used for direct Illumina sequencing. The KAPA Quantification Kit is used to measure molar concentration. The whole library should be sequenced.

Alternatively, the library can be amplified, as follows, using reaction mix shown in Table 14.

TABLE 14
Reaction mix for library amplification.

| | | |
|---|---|---|
| Reagent | Volume | Final conc. |

| 10 μM Illumina P1/P2 | 5 | μL | 1 | μM |
|---|---|---|---|---|
| primers | | | | |
| 10 mM dNTP | 1 | μL | 200 | μM |
| 5x Phusion HF buffer | 10 | μL | 1x | |
| 2 U/μL Phusion | 1 | μL | 0.04 | U/μL |
| polymerase | | | | |
| Nuclease-free water | 23 | μL | | |
| Total volume | 50 | μL | | |

The library is amplified under the following conditions: 98° C. for 30 s, 12 cycles of [98° C. 10 s, 65° C. 30 s, 72° C. 30 s], 72° C. 5 min, 4° C. The cDNA is purified on AmPure and resuspended in 40 μL. The expected concentration is around 20 nM.

Reagents and Equipment

| Component | Source |
|---|---|
| Microfluidic | Dolomite Microfluidics custom design |
| device | |
| 2x BWT | 10 mM Tris HCl pH 7.5, 1 mM EDTA, |
| | 2M NaCl, 0.02% Tween-20 |
| Thermostable | NEB M0296S |
| PPase | |
| Platinum Taq | Life Technologies 10966-026 |
| Capture Beads | Spherotech 20 μm paramagnetic streptavidin-coated |
| | polystyrene beads (custom order SVM-200-4) |
| EBT | 10 mM Tris pH 7.5, 1 mM EDTA, 0.02% Tween-20 |
| ABIL WE09 | Degussa |
| Tegosoft DEC | Degussa |
| Mineral oil | Sigma Aldrich M5904 |
| Breaking Buffer | 10 mM Tris-HCl (pH 7.5), 1% Triton-X 100, 1% SDS, |
| | 100 mM NaCl, 1 mM EDTA |
| TNT Buffer | 20 mM Tris pH 7.5, 50 mM NaCl, 0.02% Tween |
| SYBR Gold | Life Technologies |
| Melt Solution | 0.1M NaOH (prepare fresh each time) |
| Exonuclease I | NEB (BioNordika) M0293S |
| RNase H | NEB (BioNordika) M0297S |
| USER Enzyme | NEB (BioNordika) M5505S |
| CircLigase II | Epicentre (Nordic Biolabs) CL9025K |
| Custom oligos | Trilink |

### Example 2—Generating a Cell Map of the Dorsal Root Ganglion

Previously published methods have been applied to cell-type discovery in the dorsal root ganglion. A cell map was generated from 864 single-cell transcriptomes. About ten clusters were identified, which is similar to the approximately ten known cell types in this tissue. Examining known markers, it was found that clusters do indeed correspond to cell types. In FIG. 9, four clusters are shown, where each node corresponds to a single cell, and the intensity of staining from black (low) to white (high) corresponds to the expression of the proprioceptive neuron marker Parvalbumin. Expression of Parvalbumin (a marker of proprioceptive neurons) was detected only in Cluster 4, showing that this cluster indeed represents proprioceptive neurons.

### Example 3—RNA Capture and cDNA Synthesis on Barcoded Beads

Barcoded 20 μm polystyrene beads can bind RNA, and the RNA can be effectively reverse transcribed on them, the cDNA Library can then be amplified from a desired number of beads. This example shows that cDNA library prepared on barcoded beads is comparable with the one synthetized on standard 1 um streptavidin coated paramagnetic polystyrene beads (MyOne Cl Streptavidin beads) loaded by a single-stranded P1A-sequence-flanked polyT oligonucleotide (T8U_P2A-T31). These beads have a high surface-to-volume ratio so are expected to yield an optimal library. As additional comparison T8U_P2A-T31 was bound to 20 um diameter streptavidin coated polystyrene magnetic beads (SVM1-200-4 Spherotech), the same beads as the barcoded.

In an ideal situation where a single bead is compartmentalized with a cell in a volume of 25 pL, after lysis one would have an mRNA concentration of 200 ng/ul for an average sized cell. These conditions are possible to reproduce in a big volume (e.g. in a 1.5 mL tube) maintaining the concentration. In this example, however, we worked with a much lower concentration (75 ng/μl) to simulate a worst-case scenario.

Barcoded beads were prepared as in EXAMPLE 1, approximately 100,000 beads were used and resuspended directly in 20 ul LiBT.

The beads for comparison were prepared as follows: 25 ul of 1 μm diameter MyOne C1 Streptavidin coated beads (stock 10 mg/ml) or 40 ul of 20 μm diameter Streptavidin Magnetic Particles (stock 1% w/v) were resuspended in LiWT, washed tree times: twice by short centrifugation and removal of the supernatant and the third time by binding the beads using a magnetic stand.

The beads were then resuspended in 25 μL BWT 2×. Successively, 25 ul of T8U_P2A-T31 10 uM was added to the suspension and the beads were incubated to bind the oligonucleotides for 30 min. After incubation the beads were washed once in 50 ul BWT 1× and once in 60 ul LiBT and finally resuspended in 20 ul LiBT.

Human Reference total RNA (Agilent) was used as template, 20 ul of RNA was heated up at 72° C. for 2 minutes. Then the RNA was quickly transferred on ice. The beads, were resuspended in 20 ul LiBT and added to the RNA. The mix was incubated for 5 min at R.T. under agitation.

The beads were washed once with 60 ul LiWT, bound again to the magnet and resuspended in 30 ul of RT mix prepared as follows:

| | | |
|---|---|---|
| Reagent | Volume x3 | Concentration |

| Water + Tween 0.02% | 28.5 | ul | | |
|---|---|---|---|---|
| 5M Betaine | 15 | ul | 0.82M | |
| 5x SuperScript | 18 | ul | 1x | |
| First-strand buffer | | | | |
| MgCl₂[100 mM] | 5.4 | ul | 6 | mM |
| DTT [100 mM] | 4.5 | ul | 5 | mM |
| dNTPs [20 mM] | 4.5 | ul | 1 | mM |
| Superscript II [200 | 9 | ul | 20 | U/ul |
| U/μl] | | | | |
| P2A_PvuI-rTSO (40 uM) | 12 | ul | 5 | uM |
| Total volume | 90 | ul | | |

The sample was incubated in a thermal cycler with the program: step1: 1 h at 42° C. step2: 10 min at 70° C.

During this time, the beads were checked periodically for sedimentation and resuspended if necessary. Finally the beads were washed twice in 60 ul LiWT and resuspended in 35 ul LiWT.

Only a fraction (1.2 ul) of of those beads were used for the following PCR

The PCR buffer was prepared as follows:

| | | |
|---|---|---|
| Reagent | Volume x3 | Final in PCR |

| | | | |
|---|---|---|---|
| Water + Tween 0.02% | 87 | ul | |
| 10x Advantage Buffer | 11.5 | ul | 1x |
| dNTPS (20 mM) | 2.25 | ul | 400 uM |
| bio-P2A(PCR) [20 uM] | 3 | ul | 530 nM |
| Advantage Polymerase | 4.5 | ul | |

1.2 ul of the barcoded-cDNA beads (containing between 3100-3400 beads as extimated using a Bürker chamber) were added to 35 ul of PCR buffer.

The reaction was incubated in a thermal cycler set up as follows: step1: 1 min at 95° C., step2: 20 sec at 95° C., step3: 4 min at 58° C., step4: 6 min at 68° C., step5: Go to step2 4 times, step6: 20 sec at 95° C., step7: 30 sec at 64° C., step8: 6 min at 68° C., step9: Go to step2 8 times, step10: 20 sec at 95° C., step11: 30 sec at 64° C., step12: 7 min at 68° C., step13: Go to step2 3 times. step14: 10 min at 72° C.

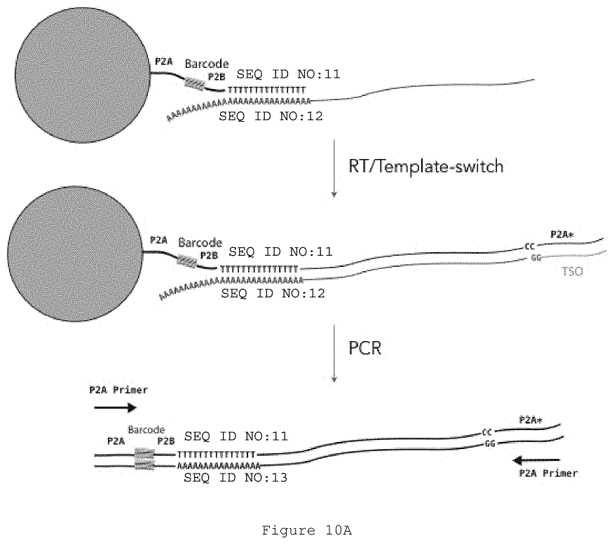

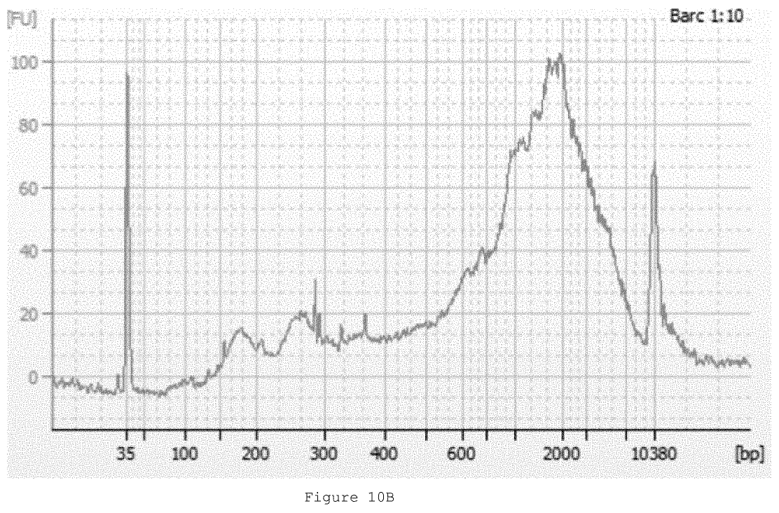

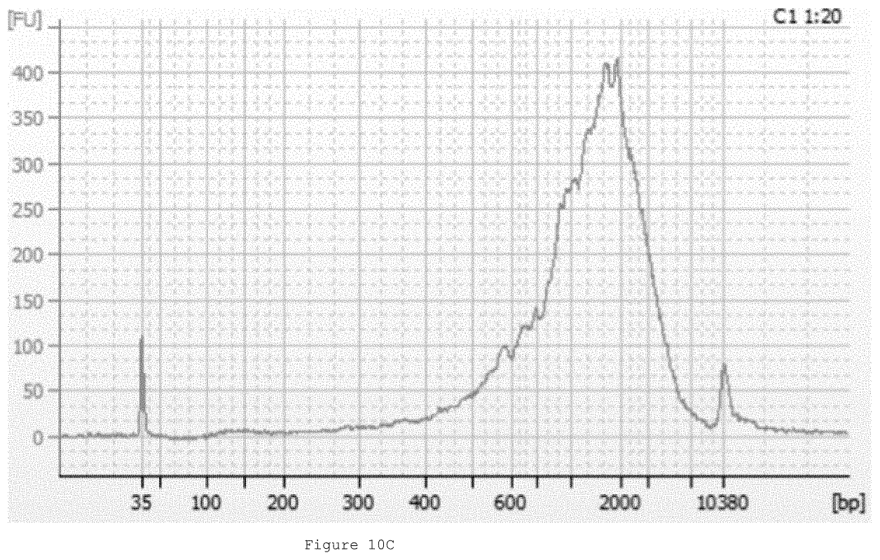

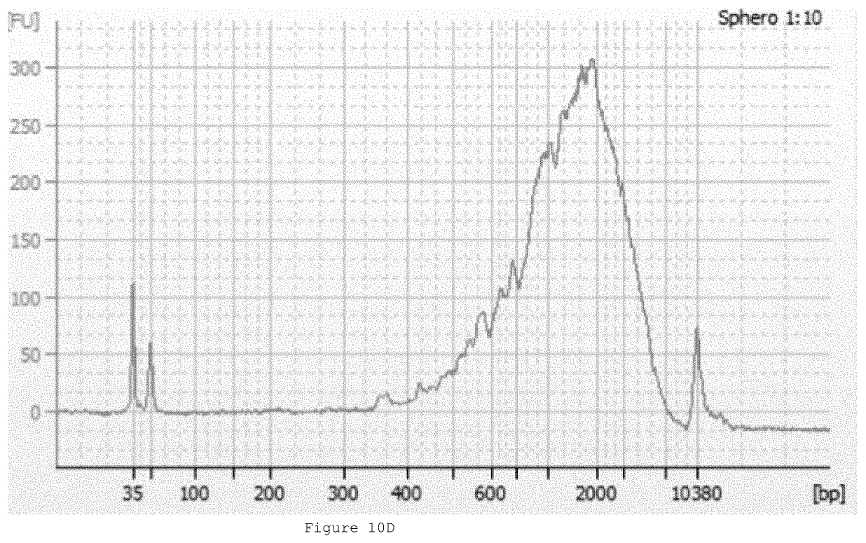

The PCR product was quantified and 3 ng was loaded on a Bioanalyzer electrophoretic system resulting in the electrophoregrams shown in FIGS. 10B to 10D.

Library Preparation for Illumina Sequencing

The cDNA library has to be converted sequencing library for Illumina sequencing by adding the other sequence (P1a) required for cluster on an Illumina flow cell. This can be done in one step by means of a Tn5 transposase-based reaction. Tn5 transposase was loaded by mixing 5 ul of 15 uM P1A-ME adapters and 5 ul of the protein 14.5 uM. The mix was incubated at 37° C. for 1 h in a shaker at 500 rpm. 90 ul of 50% glycerol were added to dilute the Tn5 to optimal concentration. The reaction was prepared in the following way:

| Reagent | Volume | |
|---|---|---|
| Amplified cDNA (diluted | 12 | μL |
| to 3.5 ng/ul) | | |
| Nuclease-free water | 45 | μL |
| TAPS buffer* | 9 | μL |
| 100% DMF | 9 | μL |
| Loaded Transposome | 11.5 | μL |
| stock | | |
| Total volume | 90 | μL |

The suspension was incubated at 55° C. for 6 minutes. To stop the reaction the tube was put on ice and Streptavidin 1 um paramagnetic beads (MyOnce C1) previously suspended in 30 ul BWT2x were added. The suspension was incubated 20 min at RT, the beads were bound to a magnet and washed three times, alternating TNT with Qiaquick PB buffer.

Since this reaction generates 5′ and 3′ fragments bound to the beads (and only the 3′ fragments are the desired target), the 5′ ends were digested and thus destroyed using PvuI restriction enzyme. This was done by resuspending the beads in the following reaction mix:

| Reagent | Volume x1 | Final conc. |
|---|---|---|
| Water 0.02% tween | 88 μL | |
| 10x CutSmart | 10 μL | 1x |
| PvuI-HF enzyme (20 | 2 μL | 0.4 U/μL |
| U/μL) | | |
| Total volume | 100 μL | |

The mix was incubated 37° C. for one hour on a shaker to avoid beads precipitation. The beads were washed three times in TNT, resuspended in 15 μl water and incubated for 10 min at 70° C. The beads were bound and discarded the supernatant, containing the eluted sequencing library was kept. The molarity was determined by real-time PCR quantification and electropherograms.

### Example 4—Sequencing Analysis and Evaluation of the Barcoding Strategy

A library prepared by using approximately 3000 barcoded beads and Human reference RNA was sequenced on an Illumina HiSeq 2000. Index reads using the primer

(SEQ ID NO: 1)
5′-AAATCGGAAGAGCACACGTCTGAACTCCAGTCAC-3′

was used to sequence the barcodes while the main read was sequenced using sequencing primer

(SEQ ID NO: 2)
5′-ACCACCGATGCGTCAGATGTGTATAAGAGACAG-3′.

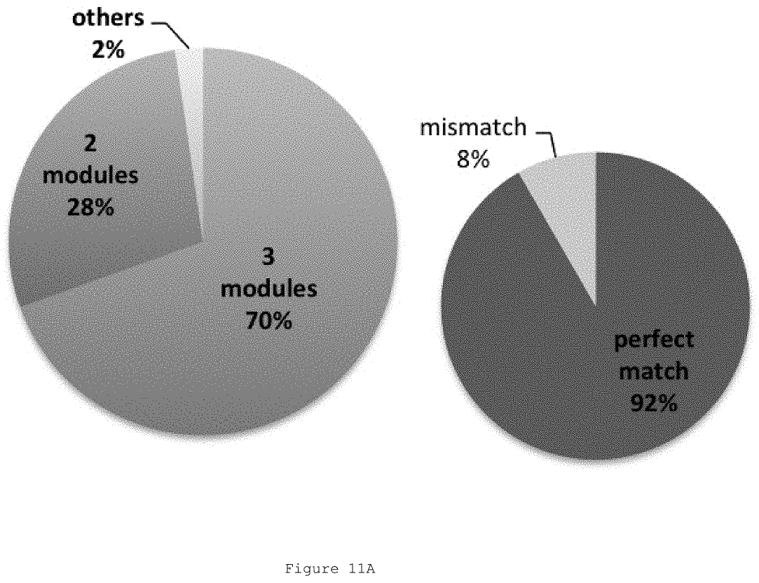

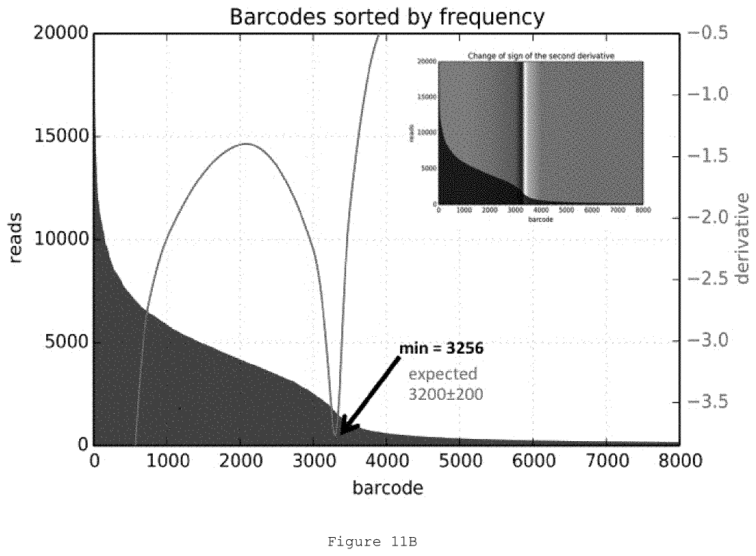

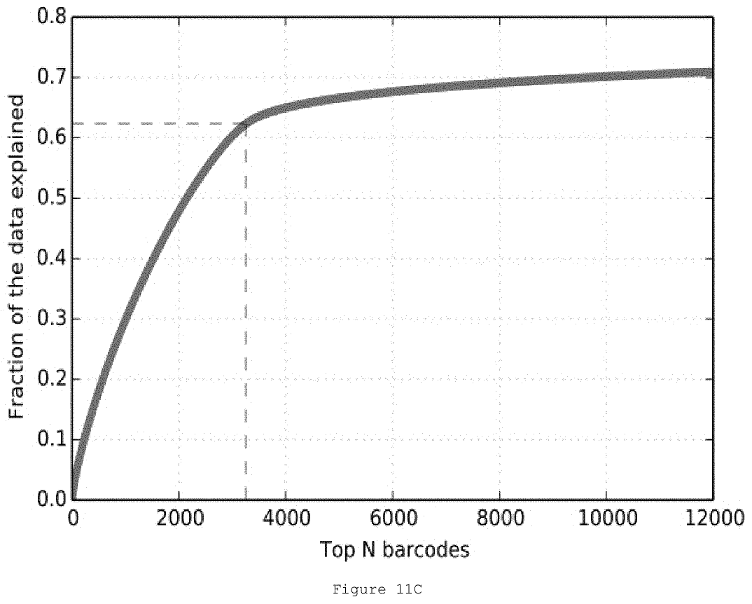

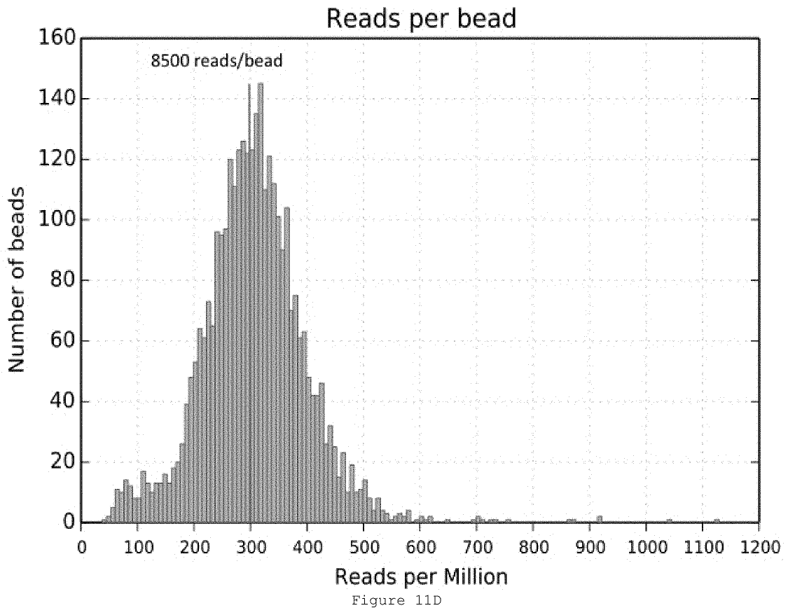

The pass-filter barcode sequences with the expected flanking regions were assigned to one of the three categories: ‘3 modules’, ‘2 modules’, ‘other’. The barcoding efficiency was assessed by counting the number of modules and evaluating the presence of mismatches in the modules (FIG. 11A). All barcodes that did not have 3-modules were discarded and the rest was used for the following analysis.

The number of sequencing reads for every barcode was counted. To distinguish real barcodes from barcodes that arise as artifacts of sequencing or PCR amplification we looked for a sudden drop of counted reads in a sorted list of barcodes, and identified it as local minimum of the first derivative. This minimum corresponded 3256th barcode, a number that strikingly fit with the number of beads used to prepare the library (about 3200; FIG. 11B). The top 3256 barcodes accounted for more than 60% of the valid reads (FIG. 11C).

We mapped against the Human Genome the transcript reads using Bowtie. Reads were then assigned to the correspondent barcode: every barcode got a number of reads ranging from 100 per million to 500 per million with an average of 300 per million (FIG. 11D).

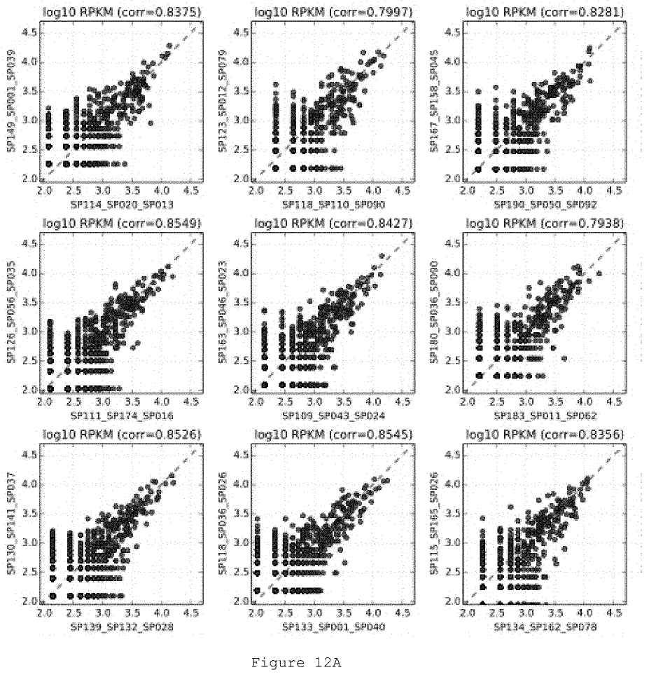

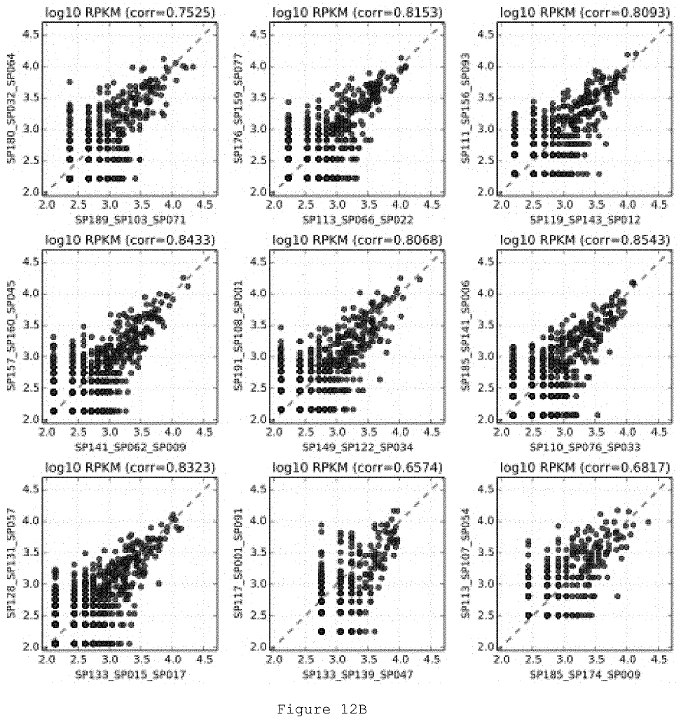

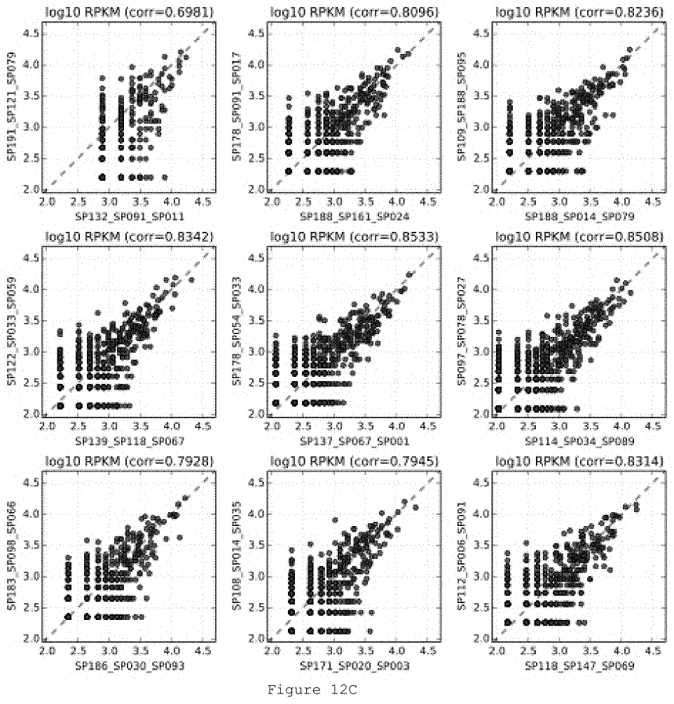

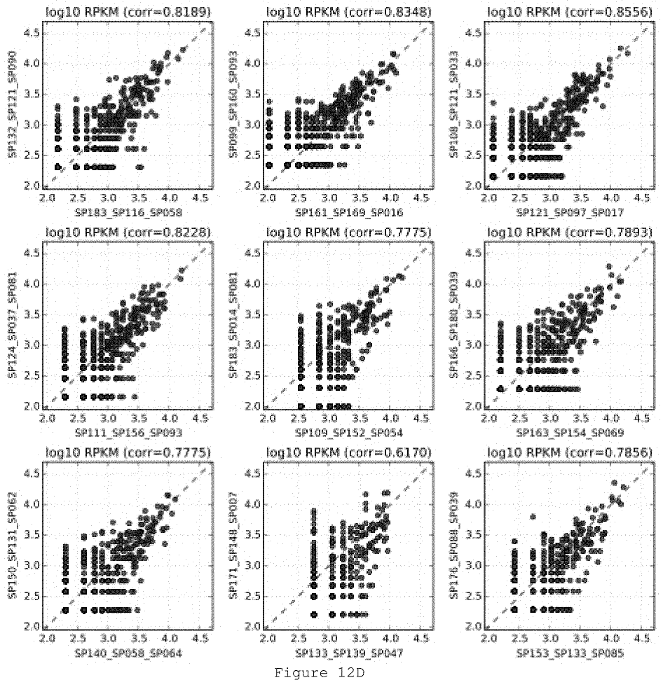

To assess the variability in capture ability of the beads we calculated the correlation coefficient and plotted scatterplots of the normalized reads (RPM) for random pairs of beads, the average correlation coefficient was 0.8 (FIGS. 12A to 12D). Furthermore the scatterplots were comparable with the one would obtain, at a comparable depth of sequencing, using state of the art low throughput single cell RNA-seq technology.

### Example 5—cDNA Synthesis in Microwell Array

In this example cells and beads are confined in the microwells where cells are lysed, RNA is captured on the beads and reverse transcribed in cDNA. The cDNA is then amplified from a selected number of beads.

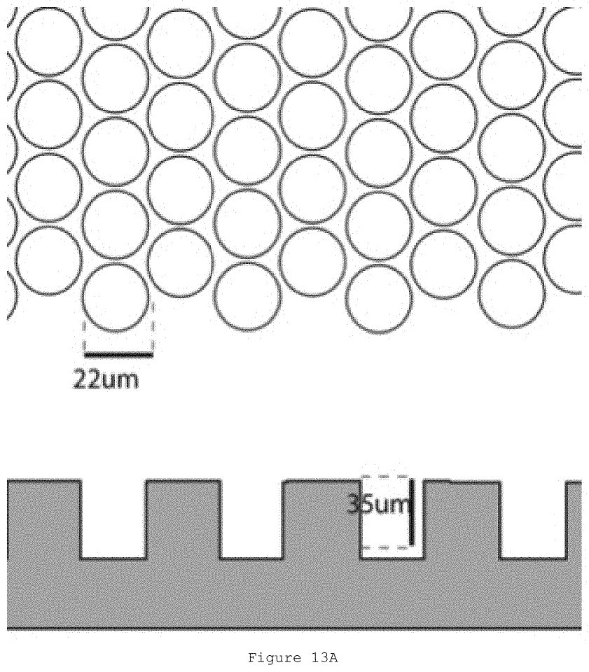

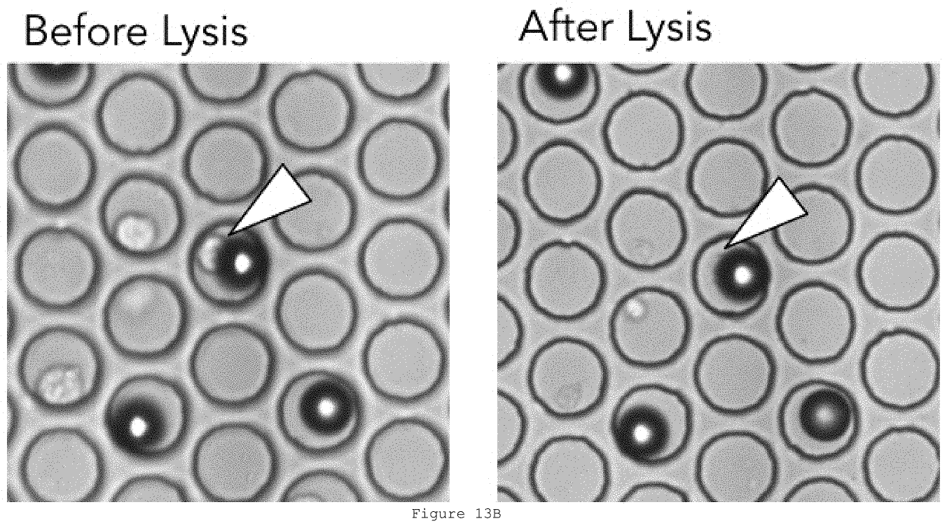

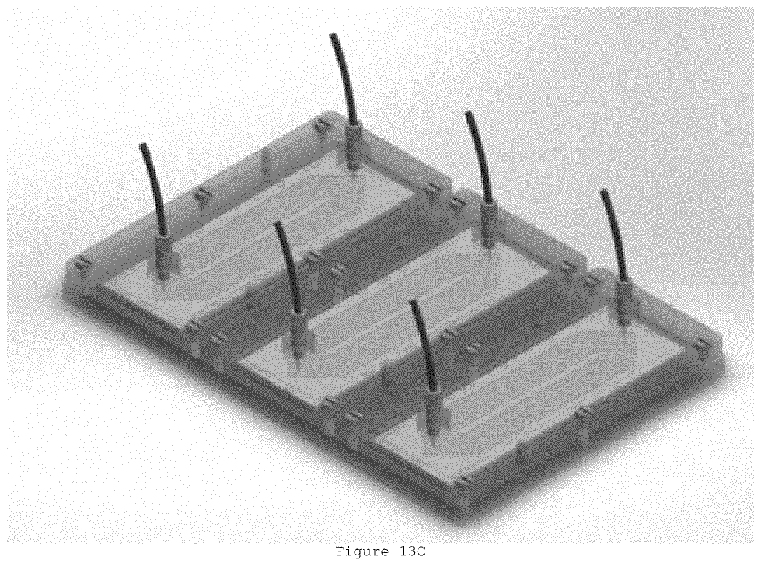

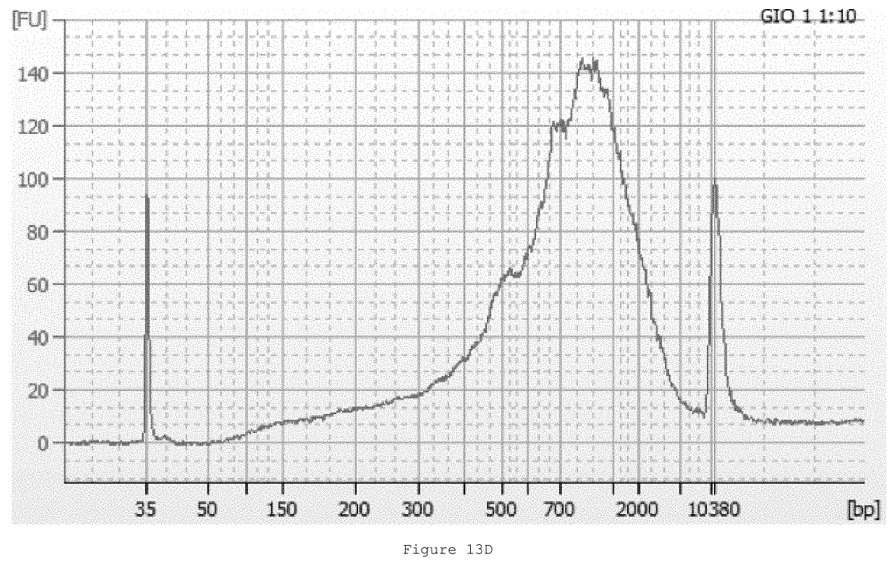

A PDMS microwell array (FIG. 13A) was manufactured using standard procedures, and assembled in a holder (FIG. 13C) to form a closed flowcell with inlet and outlet for laminar liquid flow across the surface of the array.

Cells and beads were introduced in a flowcell containing a PDMS microwell-array (FIGS. 5A and C). The ceiling of the flowcell was coated with Poly-HEMA by distributing on it evenly and only in the chamber 150 ul of Poly-HEMA (10 mg/ml in EtOH 95%) This is left on the surface for 10 minutes and washed away with water and rinsed carefully. The PDMS chip was made hydrophilic by treatment with plasma for 2 minutes. The PDMS chip was positioned and the flowcell was assembled.

To wash the PDMS chips and fill the wells 5-10 ml water was flown trough, slowly and avoiding bubbles. Successively 5 ml PBS was flown to prepare the chamber for cell loading.

500,000 cells were suspended in 600 ul cold PBS, and the cell suspension was loaded in to chamber. The cells were let sediment in the chamber for 10 minutes, the flow chamber was vortexed 4 times during this period to improve cells entry in the microwells. 250000 P2A_T31-coated 10 um beads were resuspended in 550 ul PBS and loaded in the flow cell. The beads were let sink for 8 min, vortexing 3 times in this time span. 1 ml Lysis buffer was flown through and lysis/RNA hybridization was allowed for 10 min. Microphotographs were taken before and after lysis (FIG. 13B). The chamber was washed with 250 ul LiWT followed by 250 ul Superscript First Strand buffer 1×. Chamber was then filled with the following RT buffer

| Reagent | Volume | Conc in buffer | |
|---|---|---|---|
| Water + Tween 0.02% | 171 ul | | |
| 5M Betaine | 90 ul | 0.82M | |
| 5x SuperScript | 108 ul | 1x | |
| First-strand buffer | | | |
| MgCl₂ [100 mM] | 32 ul | 6 | mM |
| DTT [100 mM] | 27 ul | 5 | mM |
| dNTPs [20 mM] | 27 ul | 1 | mM |
| Superscript II [200 | 50 ul | 20 | U/ul |
| U/μl] | | | |
| P2A_PvuI-rTSO (40 uM) | 28 ul | 5 | uM |
| Total volume | 540 ul | | |

The flow cell was placed in a water bath whose temperature was set at 45° C. and the flow cell was incubated for 2 h.

After incubation the beads were harvested. First the 2 ml 0.5×LiBT were flown into the flowcell. Then the flow cell was disassembled, the PDMS chip sliced in 5 pieces. A slice of PDMS chip was dipped in PCR buffer agitated with a pipette tips and vortexed to free the beads in the mix.

| | | |
|---|---|---|
| Reagent | Volume | Final in PCR |

| | | | |
|---|---|---|---|
| Water + Tween 0.02% | 180 | ul | |
| 10x Advantage Buffer | 23 | ul | 1x |
| dNTPS (20 mM) | 5 | ul | 400 uM |
| bio-P2A(PCR) [100 uM] | 1.3 | ul | 530 nM |
| Advantage Polymerase | 9 | ul | |

| | |
|---|---|
| Sample cDNA on | 1.75 ul per |
| Beads with LiWT | condition |

PDMS was removed and DNA polymerase (Advantage Polymerase mix) was added.

The Sample was incubated in a thermal cycler set up as follows: step1: 1 min at 95° C., step2: 20 sec at 95° C., step3: 4 min at 58° C., step4: 6 min at 68° C., step5: Go to step2 4 times, step6: 20 sec at 95° C., step7: 30 sec at 64° C., step8: 6 min at 68° C., step9: Go to step2 8 times, step10: 20 sec at 95° C., step11: 30 sec at 64° C., step12: 7 min at 68° C., step13: Go to step2 6 times. step14: 10 min at 72° C.

PCR product was quantified and 3 ng were loaded for electrophoresis (FIG. 13D)

Buffers

TNT

20 mM Tris pH 7.5, 50 mM NaCl, 0.02% Tween

LiBT

20 mM Tris-HCl (pH 7.5), 1.0 M LiCl, 2 mM EDTA, 0.02% Tween

LiWT

10 mM Tris-HCl (pH 7.5), 0.15 M LiCl, 1 mM EDTA, 0.02% Tween

Lysis Buffer

10 mM Tris pH7.5, 0.15 M LiCl, 1 mM EDTA, 1% Triton

Oligonucleotides Sequences

T8U_P2A-T31
(SEQ ID NO: 3)
5′3io-TTTTTTTTUCAAGCAGAAGACGGCATACGAGATTTTTTTTTTTT
TTTTTTTTTTTTTTTTTTTT-3′
P1A-ME adapter (2 oligos hybridized)
P1A-ME

(SEQ ID NO: 4)
5′-GAATGATACGGCGACCACCGATGCGTCAGATGTGTATAAGAGACAG-
3′
rcME
(SEQ ID NO: 5)
5′Pho-CTGTCTCTTATACACATCTGACGC

### REFERENCES

1. Arendt, D. Nature Reviews Genetics 9, 868-882 (2008).
2. Vickaryous, M. K. & Hall, B. K. Biological Reviews of the Cambridge Philosophical Society 81, 425-55 (2006).
3. Harris, T. D. et al. Science 320, 106-109 (2008).
4. Eid, J. et al. Science 323, 133-138 (2009).
5. Schadt, E., Turner, S. & Kasarskis, A. Human Molecular Genetics 19, R227-R240 (2010).
6. Casbon, J. A., Osborne, R. J., Brenner, S. & Lichtenstein, C. P. Nucleic acids research 39, e81 (2011).
7. Kivioja, T. et al. Nature Methods 9, 72-4 (2011).
8. Shiroguchi, K., Jia, T. Z., Sims, P. A. & Xie, X. S. Proceedings of the National Academy of Sciences of the United States of America (2012).
9. Fu, G. K., Hu, J., Wang, P. H. & Fodor, S. P. Proceedings of the National Academy of Sciences of the United States of America 108, 9026-31 (2011).
10. Kinde, I., Wu, J., Papadopoulos, N., Kinzler, K. W. & Vogelstein, B. Proceedings of the National Academy of Sciences of the United States of America 108, 9530-5 (2011).
11. Eberwine, J. et al. Proceedings of the National Academy of Sciences of the United States of America 89, 3010-4 (1992).
12. Hashimshony, T., Wagner, F., Sher, N. & Yanai, I. Cell reports 2, 666-73 (2012).
13. Klein, C. A. et al. Nature biotechnology 20, 387-92 (2002).
14. Kurimoto, K. et al. Nucleic Acids Research 34, e42 (2006).
15. Tang, F. et al. Nat Methods 6, 377-82 (2009).
16. Maleszka, R. & Stange, G. Gene 202, 39-43 (1997).
17. Islam, S. et al. Genome Research 21, 1160-7 (2011).
18. Goetz, J. J. & Trimarchi, J. M. Nature biotechnology 30, 763-5 (2012).
19. Johnston, I. G. et al. PLoS computational biology 8, e1002416 (2012).
20. Raj, A et al PLoS Biol 4, e309 (2006).
21. Raj, A. & Vanoudenaarden, A. Cell 135, 216-226 (2008).
22. Endele, M. & Schroeder, T. Annals of the New York Academy of Sciences 1266, 18-27 (2012).

1: A method for capturing and encoding nucleic acid from a plurality of single cells, wherein the method comprises:
(i) randomly placing a plurality of solid supports into a plurality of compartments, such that the average number of solid supports per compartment, λ₁, is less than 1, wherein each solid support carries (a) a unique identification sequence and (b) a capture moiety;
(ii) randomly placing a plurality of single cells into the plurality of compartments, such that the average number of cells per compartment, λ₂, is less than 1;
(iii) releasing nucleic acid from each single cell; and
(iv) capturing the nucleic acid from each single cell via the capture moiety, such that nucleic acid from each single cell is tagged with a unique identification sequence,
wherein steps (i) and (ii) may be performed in any order.

2: The method according to claim 1, wherein the average number of solid supports per compartment, λ₁, and the average number of single cells per compartment, λ₂, are selected such that 2/(1+λ₁)(2+λ₂)≥90%.

3: The method according to claim 2, wherein λ₁ and λ₂ are selected such that 2/(1+λ₁)(2+λ₂)≥95%.

4: The method according to claim 1, wherein the plurality of solid supports comprising (a) a unique identification sequence and (b) a capture moiety are generated prior to step (ii) by emulsion PCR.

5: The method according to claim 1, wherein the plurality of solid supports are generated prior to step (ii) by split-and-pool combinatorial synthesis.

6: The method according to claim 1, wherein the plurality of compartments are wells of a microwell array.

7: The method according to claim 1, wherein the plurality of compartments are droplets formed by an emulsifying or droplet microfluidics apparatus.

8: The method according to claim 1, wherein the compartment volume is selected such that only a single solid support can fit into each compartment.

9: The method according to claim 1, wherein the solid support is a microbead.

10: The method according to claim 1, wherein the unique identification sequence is an oligonucleotide.

11: The method according to claim 1, wherein the capture moiety is a nucleic acid complementary to cellular nucleic acid.

12: The method according to claim 11, wherein the unique identification sequence and the capture moiety are both nucleic acid sequences and are part of the same oligonucleotide.

13: The method according to claim 1, wherein each solid support carries a plurality of different capture moieties.

14: The method of claim 13, wherein the unique identification sequence and the capture moiety are both nucleic acid sequences and are part of the same oligonucleotide and wherein the solid support carries a plurality of oligonucleotides, each comprising a unique identification sequence and a different capture moiety.

15: The method according to claim 1, wherein the nucleic acid to be captured and encoded is RNA, such as mRNA, rRNA, tRNA, ncRNAs, mitochondrial RNA; nuclear or mitochondrial DNA; or microbial or viral RNA or DNA.

16: The method according to claim 1, wherein after step (iv), the method comprises the step of synthesising cDNA from the captured nucleic acid.


SEQUENCE LISTING (17 sequences)

SEQ ID NO: 1 - DNA, 34 bp, Artificial Sequence
  Description of Artificial Sequence Synthetic primer
aaatcggaagagcacacgtctgaactccagtcac

SEQ ID NO: 2 - DNA, 33 bp, Artificial Sequence
  Description of Artificial Sequence Synthetic primer
accaccgatgcgtcagatgtgtataagagacag

SEQ ID NO: 3 - DNA, 64 bp, Artificial Sequence
  Description of Artificial Sequence Synthetic oligonucleotide
  Description of Combined DNA/RNA Molecule Synthetic oligonucleotide
ttttttttucaagcagaagacggcatacgagatttttttttttttttttttttttttttttttt

SEQ ID NO: 4 - DNA, 46 bp, Artificial Sequence
  Description of Artificial Sequence Synthetic oligonucleotide
gaatgatacggcgaccaccgatgcgtcagatgtgtataagagacag

SEQ ID NO: 5 - DNA, 24 bp, Artificial Sequence
  Description of Artificial Sequence Synthetic oligonucleotide
ctgtctcttatacacatctgacgc

SEQ ID NO: 6 - DNA, 33 bp, Artificial Sequence
  Description of Artificial Sequence Synthetic oligonucleotide
  Description of Combined DNA/RNA Molecule Synthetic oligonucleotide
ttttttttucaagcagaagacggcatacgagat

SEQ ID NO: 7 - DNA, 35 bp, Artificial Sequence
  Description of Artificial Sequence Synthetic oligonucleotide
ccatctcgtatgccgtcttctgcttgaaaaaaaaa

SEQ ID NO: 8 - DNA, 34 bp, Artificial Sequence
  Description of Artificial Sequence Synthetic oligonucleotide
ggtgactggagttcagacgtgtgctcttccgatc

SEQ ID NO: 9 - DNA, 27 bp, Artificial Sequence
  Description of Artificial Sequence Synthetic oligonucleotide
gaagagcacacgtctgaactccagtca

SEQ ID NO: 10 - DNA, 34 bp, Artificial Sequence; identical to SEQ ID NO: 8
  Description of Artificial Sequence Synthetic oligonucleotide

SEQ ID NO: 11 - DNA, 15 bp, Artificial Sequence
  Description of Artificial Sequence Synthetic oligonucleotide
ttttttttttttttt

SEQ ID NO: 12 - DNA, 28 bp, Artificial Sequence
  Description of Artificial Sequence Synthetic oligonucleotide
aaaaaaaaaaaaaaaaaaaaaaaaaaaa

SEQ ID NO: 13 - DNA, 16 bp, Artificial Sequence
  Description of Artificial Sequence Synthetic oligonucleotide
aaaaaaaaaaaaaaaa

SEQ ID NO: 14 - DNA, 43 bp, Artificial Sequence
  Description of Artificial Sequence Synthetic oligonucleotide
  Description of Combined DNA/RNA Molecule Synthetic oligonucleotide
  modified_base (36)..(41): a, c, t, g, unknown or other
ttttttttucaagcagaagacggcatacgagatggnnnnnngg

SEQ ID NO: 15 - DNA, 43 bp, Artificial Sequence
  Description of Artificial Sequence Synthetic oligonucleotide
  modified_base (3)..(8): a, c, t, g, unknown or other
ccnnnnnnccatctcgtatgccgtcttctgcttgaaaaaaaaa

SEQ ID NO: 16 - DNA, 41 bp, Artificial Sequence
  Description of Artificial Sequence Synthetic oligonucleotide
  Description of Combined DNA/RNA Molecule Synthetic oligonucleotide
  modified_base (36)..(41): a, c, t, g, unknown or other
ttttttttucaagcagaagacggcatacgagatggnnnnnn

SEQ ID NO: 17 - DNA, 10 bp, Artificial Sequence
  Description of Artificial Sequence Synthetic oligonucleotide
  modified_base (3)..(8): a, c, t, g, unknown or other
ggnnnnnngg